(19) United States
(12) Patent Application Publication
(10) Pub. No.: US 2023/0289955 A1
(43) Pub. Date: Sep. 14, 2023

(54) END-TO-END PART LEARNING FOR IMAGE ANALYSIS

(71) Applicant: MEMORIAL SLOAN KETTERING CANCER CENTER, New York, NY (US)

(72) Inventors: Chensu XIE, New York, NY (US); Hassan MUHAMMAD, New York, NY (US); Chad M. VANDERBILT, New York, NY (US); Raul CASO, New York, NY (US); Dig Vijay Kumar YARLAGADDA, New York, NY (US); Gabriele CAMPANELLA, New York, NY (US); Thomas J. FUCHS, New York, NY (US)

(73) Assignee: MEMORIAL SLOAN KETTERING CANCER CENTER, New York, NY (US)

(21) Appl. No.: 18/008,133

(22) PCT Filed: Jun. 2, 2021
(86) PCT No.: PCT/US2021/035443

§ 371 Date: Dec. 2, 2022

Related U.S. Application Data
(60) Provisional application No. 63/033,733, filed on Jun. 2, 2020.

(51) Int. Cl. G06T 7/00; G06V 10/44; G06V 10/762; G06V 10/764; G16H 30/40

(52) U.S. Cl. CPC G06T 7/0012; G06V 10/44; G06V 10/762; G06V 10/764; G16H 30/40; G06T 2207/30024

(57) ABSTRACT

Presented herein are systems and methods of classifying biomedical images. A computing system may identify a first plurality of tiles from a first biomedical image of a first sample. The computing system may determine a first category for the first sample by applying the plurality of tiles to a classification model. The classification model may include a tile encoder to determine, based on the first plurality of tiles, a corresponding plurality of feature vectors in a feature space. The classification model may include a clusterer to select a subset of feature vectors from the plurality of feature vectors based on a plurality of centroids defined in the feature space. The classification model may include an aggregator to generate, based on the subset of feature vectors, the first category for the sample. The computing system may store an association between the first category and the first biomedical image.

### CROSS REFERENCE TO RELATED APPLICATIONS

The present application claims priority to U.S. Provisional Application No. 63/033,733, titled “End-to-End Part Learning for Image Analysis,” filed Jun. 2, 2020, which is incorporated herein by reference in its entirety.

### BACKGROUND

Computer vision algorithms may be used to recognize and detect various objects and features on digital images.

### SUMMARY

An emerging technology in cancer care and research is the use of histopathology whole slide images (WSI). Leveraging computation methods to aid in WSI assessment poses unique challenges. WSIs, being extremely high resolution giga-pixel images, cannot be directly processed by convolutional neural networks (CNN) due to huge computational cost. For this reason, other methods for WSI analysis adopt a two-stage approach where the training of a tile encoder is decoupled from the tile aggregation. This results in a trade-off between learning diverse and discriminative features. In contrast, presented herein is end-to-end part learning (EPL) which is able to learn diverse features while ensuring that learned features are discriminative. Each WSI is modeled as comprising k groups of tiles with similar features, defined as parts. A loss with respect to the slide label is back-propagated through an integrated CNN model to k input tiles that are used to represent each part. These experiments show that EPL is capable of clinical grade prediction of prostate and basal cell carcinoma. Further, it is shown that diverse discriminative features produced by EPL succeeds in multi-label classification of lung cancer architectural subtypes. Beyond classification, this method provides rich information of slides for high quality clinical decision support.

Aspects of the present disclosure are directed to systems and methods of training models to classifying biomedical images. A computing system may identify a training dataset comprising (i) a plurality of tiles of a biomedical image of a sample and (ii) a label identifying a first category for the sample. The computing system may apply the plurality of tiles to a classification model. The classification may include a tile encoder having a first plurality of weights to generate, based on the plurality of tiles, a corresponding plurality of feature vectors defined in a feature space. The classification may include a clusterer to select a subset of feature vectors from the plurality of feature vectors based on a plurality of centroids defined in the feature space. The classification may include an aggregator having a second plurality of weights to determine, based on the subset of feature vectors, a second category for the sample. The computing system may determine a loss metric based on a comparison between the second category determined by the classification model with the first category of the label of the training dataset. The computing system may update, using the loss metric, at least one of the first plurality of weights in the tile encoder, the plurality of centroids of the clusterer, or the second plurality of weights in the aggregator based on the comparison. The computing system may store, in one or more data structures, the first plurality of weights in the tile encoder, the plurality of centroids defined by the clusterer, and the second plurality of weights of the aggregator.

In some embodiments, the computing system may apply the plurality of tiles from the biomedical image of a plurality of biomedical images to the classification model. The clusterer of the classification model may identify the plurality of feature vectors from which to select the subset of feature vectors based on the biomedical image.

In some embodiments, the computing system may apply the plurality of tiles to the classification model. The clusterer of the classification model may select a subset of tiles in the biomedical image based on the subset of feature vectors.

In some embodiments, the computing system may apply the plurality of tiles to the classification model. The aggregator of the classification model may determine a plurality of confidence scores for a subset of tiles corresponding to the subset of feature vectors.

In some embodiments, the computing system may identify the training dataset comprising a plurality of labels for a corresponding first plurality of categories for the sample. In some embodiments, the computing system may apply the plurality of tiles to the classification model. The aggregator of the classification model may determine a second plurality of categories for the sample.

In some embodiments, the computing system may determine a second loss metric based on comparison among the plurality of feature vectors and the plurality of centroids in the feature space. In some embodiments, the computing system may update, using the second loss metric, at least one of the first plurality of weights in the tile encoder or the plurality of centroids of the clusterer within the feature space.

In some embodiments, the computing system may determine the plurality of centroids based on a second plurality of feature vectors generated by the tile encoder, subsequent to updating of the first plurality of weights.

Aspects of the present disclosure are directed to systems and methods of classifying biomedical images. A computing system may identify a first plurality of tiles from a first biomedical image of a first sample. The computing system may determine a first category for the first sample by applying the plurality of tiles to a classification model. The classification model may be trained using a training dataset. The training dataset may have a plurality of examples each including (i) a second plurality of tiles of a second biomedical image of a second sample and (ii) a label identifying a second category for the sample. The classification model may include a tile encoder having a first plurality of weights to determine, based on the first plurality of tiles, a corresponding plurality of feature vectors in a feature space. The classification model may include a clusterer to select a subset of feature vectors from the plurality of feature vectors based on a plurality of centroids defined in the feature space. The classification model may include an aggregator having a second plurality of weights to generate, based on the subset of feature vectors, the first category for the sample. The computing system may store an association between the first category and the first biomedical image.

In some embodiments, the computing system may provide the association between the first category and the first biomedical image. In some embodiments, the computing system may determine a plurality of confidence scores for a subset of tiles corresponding to the subset of feature vectors. In some embodiments, the computing system may determine a second plurality of categories for the sample.

In some embodiments, the computing system may select a subset of tiles in the biomedical image based on the subset of feature vectors. In some embodiments, the computing system may select the first plurality of tiles from a second plurality of tiles of the first biomedical image. In some embodiments, the computing system may obtain the first biomedical image of the first sample via a histological image preparer.

### BRIEF DESCRIPTION OF THE DRAWINGS

FIG. 1: Overview of the proposed end-to-end part-learning (EPL) for WSI analysis. All tile encoder θₑ's share parameters. a) Manifold initialization: randomly assign the tiles of the whole dataset to k groups, randomly initialize θₑ, and calculate the global centroids {z₁, . . . , zₖ} of each group in feature space. b) Map tiles of a training slide Si to the k clusters in the manifold (dashed regions). c) Approximate the slide specific centroids {z₁ⁱ, . . . , zₖⁱ} by their nearest tiles {xᵢ¹, . . . , xₖⁱ}. Feed the k tiles to model for slide label prediction. d) Push tiles of each part to the corresponding slide specific centroids by mean square error (MSE). e) The 2 objectives are trained concurrently. The manifold defined by θₑ changes during training. Tiles will be re-assigned to new centroids by a k-means step every n iterations.

FIGS. 2A and 2B: Convergence: EPL vs EPL with no feature attribution (EPL-NA). The prostate cancer classification training with l=512 converges better with feature attribution.

FIG. 3: Part attribution and the centroid approximation tiles used by EPL for prostate and BCC cancer classification. Each row corresponds to a part. Each column represents a test slide and the 8 tiles used for its classification. The heatmap bar represents the part attribution. 1's and 0's on top means if it's cancer slide or not. A black square means that no tiles of this slide were assigned to this part. The parts with high attribution for both tasks are basically the groups of cancer tiles (from above, left: row 7,8; right: row 1,7,8). For these parts, the negative slides either contain no tiles belonging to them (black squares) or have non-tumor tiles similar in appearance to tumor. EPL combined different morphological subtypes of tumors for the final prediction.

FIG. 4: From top to bottom: 1. Green ink. 2. Red blood cells in blood vessels near alveolar spaces. 3. Macrophages in alveolar spaces, often with hemosiderin in the macrophages. 4. Normal alveolar wall. 5. Cancer enriched for micropapillary subtype. 6. Cancer enriched for acinar subtype. 7. Black ink. 8. Cancer enriched for lepidic subtype. 9. Cancer enriched for high grade morphology, solid like. 10. Blood vessel and alveolar wall with sparse cells in spaces. 11. Cancer enriched for papillary subtype. 12. Stroma.

FIG. 5: Mapping of the learned parts back onto a prostate needle biopsy. Each part is linked to an importance score (attribution). Colors were randomly chosen and irrelevant to attribution. The tiles to be highlighted for clinical decision support can be filtered by their distance to the part centroids.

FIG. 6 is a block diagram depicting a system for classifying biomedical images using machine learning models in accordance with an illustrative embodiment.

FIG. 7 is a block diagram of a training process in the system for classifying biomedical images using machine learning models in accordance with an illustrative embodiment.

FIGS. 8A and 8B are block diagrams of an architecture for a classification model in the system for classifying biomedical images using machine learning models in accordance with an illustrative embodiment.

FIG. 9A is a block diagram of an architecture of an encoder block used to implement the classification model in the system for classifying biomedical images using machine learning models in accordance with an illustrative embodiment.

FIG. 9B is a block diagram of an architecture of a convolution stack of the encoder block used to implement the classification model in the system for classifying biomedical images using machine learning models in accordance with an illustrative embodiment.

FIG. 10 is a block diagram of an updating process in the system for classifying biomedical images using machine learning models in accordance with an illustrative embodiment.

FIG. 11 is a block diagram of an inference process in the system for classifying biomedical images using machine learning models in accordance with an illustrative embodiment.

FIG. 12A is a flow diagram depicting a method of training a model to classify biomedical images using machine learning models in accordance with an illustrative embodiment.

FIG. 12B is a flow diagram depicting a method of classifying biomedical images using machine learning models in accordance with an illustrative embodiment.

FIG. 13 is a block diagram of a server system and a client computer system in accordance with an illustrative embodiment.

### DETAILED DESCRIPTION

Following below are more detailed descriptions of various concepts related to, and embodiments of, systems and methods for maintaining databases of biomedical images. It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes.
Section A describes whole slide tissue histopathology analysis by end-to-end part learning;
Section B describes systems and methods of classifying biomedical image using machine learning models; and
Section C describes a network environment and computing environment which may be useful for practicing various embodiments described herein.

### A. Whole Slide Tissue Histopathology Analysis by End-to-End Learning

Other methods for WSI analysis adopt a two-stage approach. First, by training a CNN model on small image tiles sampled from WSIs at high resolution, each tile is encoded to a prediction score or a feature vector of low dimension. Second, an aggregation model is learned to integrate the obtained tile level information for whole slide prediction. For the first-stage tile encoder training, early works utilized extra supervision from pathologists beyond slide labels. These annotations are usually extensive, pixel-wise labels, which are difficult to obtain for large datasets due to the time-consuming process for pathologists who already have high workload on service.

Conclusively, weakly-supervised approaches optimized for simple slide level labels are of high interest. One track of weakly-supervised methods utilize unsupervised techniques to learn a tile encoder. The other track of these weakly-supervised methods adopt the multiple instance learning (MIL) framework which trains the tile encoder by iteratively selecting tiles based on their encoded feature or score. By labeling the selected tiles with the slide classification target for the tile encoder training, MIL approaches aim to identify the most discriminative tiles for cancer classification, while the unsupervised approaches tend to incorporate diverse tile groups across the entire dataset for more complicated tasks such as survival analysis.

In contrast to the aforementioned two-stage approaches, this proposed method learns the WSI prediction in an end-to-end manner by modeling each WSI as comprising k groups of tiles with similar features, defined as parts. The loss with respect to the slide label is backpropagated directly through an integrated CNN model to k input tiles, representing each part. This model performs WSI analysis using end-to-end learning with an integrated CNN model. This method is referred herein as End-to-end Part Learning (EPL). It is shown that EPL is capable of clinical grade classification of prostate and basal cell carcinoma, and the diverse discriminative features learned by it are capable of multi-label prediction of lung cancer architectural subtypes. Beyond WSI classification, EPL can also provide rich information about WSIs for high quality clinical decision support, such as tissue type localization and region importance scoring. In addition, EPL may be theoretically considered to be applicable to survival regression, treatment recommendation, or any other learnable WSI label predictions.

### 1. Introduction

Histopathology analysis is a fundamental step in the pipeline of cancer care and research which includes diagnosis, prognosis, treatment selection, and subtyping. Recent years saw an increase in the digitization of histopathology whole slide images (WSI) and development of computational methods for WSI analysis. WSIs, being extremely high resolution giga-pixel images, face various challenges on their analysis. Inputting a WSI at highest resolution directly through a convolutional neural network (CNN) for training is impossible due to huge computational cost. To match input sizes for feed-forward CNN models, a WSI would need to be down-sampled by a factor of ˜100×, resulting in the loss of cellular and structural details which are critical for prediction.

To overcome this bottleneck, other methods for WSI analysis adopt a two-stage approach. First, by training a CNN model on small image tiles sampled from WSIs at high resolution, each tile is encoded to a prediction score or a feature vector of low dimension. Second, an aggregation model is learned to integrate the obtained tile level information for whole slide prediction. For the first-stage tile encoder training, early works utilized extra supervision from pathologists beyond slide labels. These annotations are usually extensive, pixel-wise labels, which are difficult to obtain for large datasets due to the time-consuming process for pathologists who already have high workload on service.

Conclusively, weakly-supervised approaches optimized for simple slide level labels are of high interest. One track of weakly-supervised methods utilize unsupervised techniques to learn a tile encoder. The other track of these weakly-supervised methods adopt the multiple instance learning (MIL) framework which trains the tile encoder by iteratively selecting tiles based on their encoded feature or score. By labeling the selected tiles with the slide classification target for the tile encoder training, MIL approaches aim to identify the most discriminative tiles for cancer classification, while the unsupervised approaches tend to incorporate diverse tile groups across the entire dataset for more complicated tasks such as survival analysis.

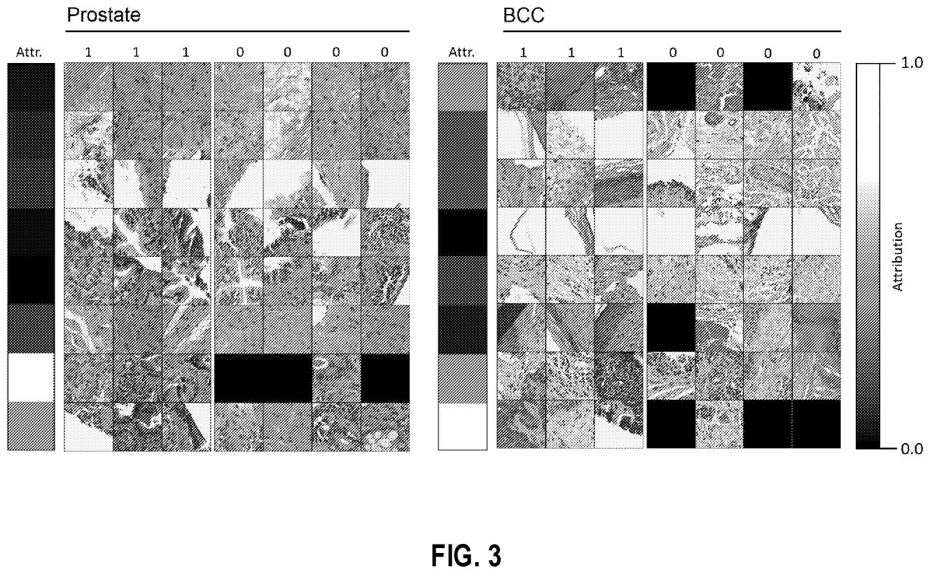

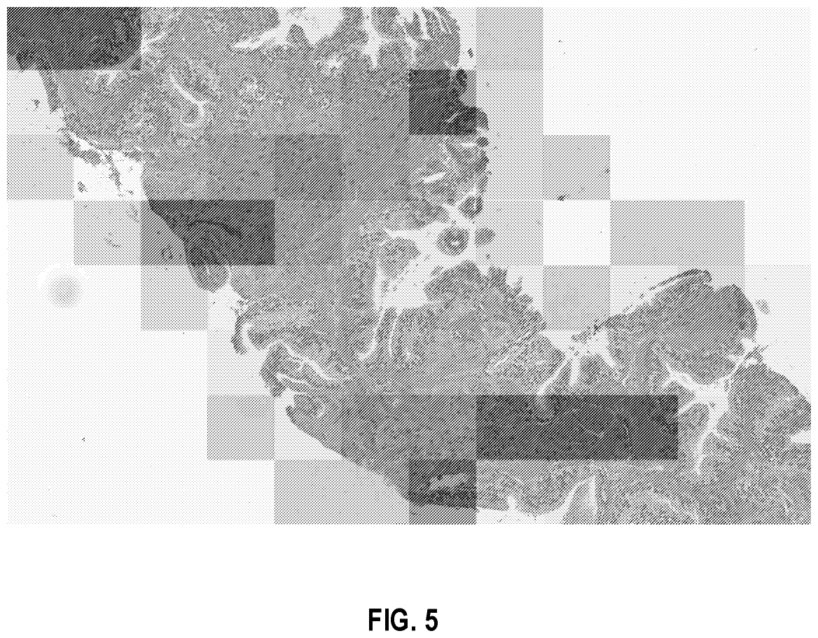

In contrast to the aforementioned two-stage approaches, the proposed method learns the WSI prediction in an end-to-end manner by modeling each WSI as comprising k groups of tiles with similar features, defined as parts. The loss with respect to the slide label is backpropagated directly through an integrated CNN model to k input tiles, representing each part (Section 3.2). This is the first model which per-forms WSI analysis using end-to-end learning with an integrated CNN model. This method is referred herein as End-to-end Part Learning (EPL). It is shown that EPL is capable of clinical grade classification of prostate and basal cell carcinoma (Section 4.1), and the diverse discriminative features learned by it are capable of multi-label prediction of lung cancer architectural subtypes (Section 4.2). Beyond WSI classification, EPL can also provide rich information about WSIs for high quality clinical decision support, such as tissue type localization (cf. FIG. 5) and region importance scoring (cf. FIG. 3 heatmap bar). In addition, EPL may be theoretically considered to be applicable to survival regression, treatment recommendation, or any other learnable WSI label predictions.

### 2. Other Approaches

### 2.1. Multiple Instance Learning for WSI Classification

Multiple instance learning approaches are popular for weakly-supervised WSI classification. In many cases of binary classification, such as predicting whether a WSI contains tumor, a positive WSI can be completely represented by a few tiles, while assuming all tiles of the negative slides can be labeled negative during training. Since this assumption does not hold for multi-class cancer type prediction, a generalized MIL approach may be applied that selects training tiles based on their prediction score of each class. It was proposed that classifying tissue images by learning the model attention to all tiles followed by weighted averaging of the tile features. Unfortunately, this method has to be combined with other tile sampling techniques to be feasible on WSIs.

### 2.2. Unsupervised Tile Encoding for WSI Analysis

MIL approach regards a tile to be predictive for a slide class if its encoded score for that class is high. However for WSI regression problems, the tile scores or features are related to the slide label in a more complicated way. Unsupervised learning of the tile encoder with constraints can produce a collection of diverse information over a WSI for the regression targets. Tile features may be learned by patch reconstruction and PCA of raw image signals respectively, constructing clusters of tiles for survival analysis. A visual dictionary of tile features may be learned for histopathological image classification. WSIs may be drastically compressed by replacing tiles with their features from contrastive learning in position before classifying the compressed slides. The proposed method EPL was motivated for learning diverse tile clusters in an end-to-end supervised way for complicated WSI analysis tasks.

### 3. Method

### 3.1. Two-Stage Methods for WSI Classification

As WSIs are too large to be trained by a CNN end-to-end, two-stage methods tend to compress a slide to a latent variable in low dimensional space by sampling tiles and passing them through a tile encoder θₑ:Z→Y, to later predict the slide label by optimizing an aggregation model θₐ:Z→Y. Given extra tile-level annotations, Z is usually the ground truth score of sampled tiles X However, for weakly supervised WSI classification with only slide level labels, learning Z is not trivial.

Methods adopting unsupervised techniques to learn Z decouple the training of θₐ and θₑ. MTh approaches model Z=Y as “pseudo labels” for tile encoder training and iteratively select predictive tiles by thresholding Z. Since Z=Y, this expectation-maximization approach can learn more discriminative features. However, it incorporates noise during the tile encoder training. The problem is aggravated in WSI regression where Z=Y might not hold for any single tile.

### 3.2. WSI Analysis by End-To-End Part Learning

Ideally, WSI prediction should be learned by end-to-end optimization of all parameters including θₐ and θₑ in an integrated CNN model based on all tiles of each slide. Formally,

maximize P( [symbol] |θₐ,θₑ,X) (1)

As using all tiles creates a huge computing graph, each WSI may be represented by k groups of tiles, each called a “part” of the slide. Since the tissue connectivity varies largely across slides (needle biopsies, large/small excisions, etc.), these parts are not defined spatially. Tiles of a slide Sᵢ are assigned to k global centroids {z₁, . . . , zₖ} of tile features θₑ(X) of the whole dataset {S₁, S₂, . . . }. Then the slide specific feature centroids {z₁ⁱ, . . . , zₖⁱ} are used to represent its k parts and connected to the aggregation module:

maximize P( [symbol] |θₐ,Z) where Z={z₁ⁱ, . . . ,zₖⁱ},zₖⁱ=1/NΣₙ₌₁^{N}θₑ(x_{k,n}ⁱ) (2)

Although the size of θₐ has been reduced largely by only taking in k centroid features, the centroids still need to be computed through a huge graph by averaging all tile features of each part. To relax the problem, each centroid may be approximated by randomly sampling one of its p nearest tiles, while maximizing the likelihood that the encoded feature of the sampled tile is equal to the corresponding centroid. By centroid approximation, the problem is reduced to a remarkably smaller model:

maximize P( [symbol] |θₐ,θₑ,{x₁ⁱ, . . . ,xₖⁱ}+P(Z|θₑ,{x₁ⁱ, . . . ,xₖⁱ}) (3)

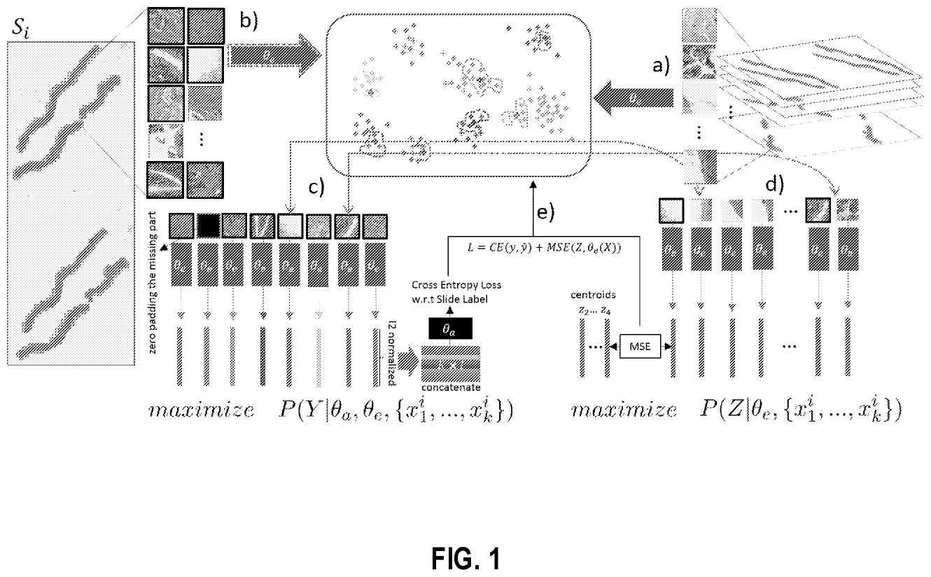

Note that now the whole model can be optimized directly from end-to-end to learn discriminative features for prediction of Y, while defining k parts with diverse features for a better representation and understanding of the whole slides. FIG. 1 is an illustration of the proposed end-to-end part learning model.

### 3.3. Manifold Initialization and Training Iterations

To initialize the data manifold, tiles of the whole training dataset are randomly split into k groups at the beginning. Parameters of θₑ are also initialized randomly. Then, the initial global centroids {z₁, . . . , zₖ}⁰ by zₖ=1/NΣₙ₌₁^{N}θₑ(x_{k,n}) (cf. FIG. 1a) may be calculated. To save computing time, 10% of training slides and 100 tiles per slide may be sub-sampled for each epoch. For a training slide Sᵢ, its 100 tiles Xⁱ are assigned to the k clusters according to their distance to the global centroids in feature space (cf. FIG. 1b). The average feature of the tiles from Si (cf. dashed regions in FIG. 1) in each cluster is the slide specific centroid for that part: zₖⁱ=1/NΣₙ₌₁^{N}θₑ(x_{k,n}ⁱ). To represent each part, the p nearest tiles to its centroid zₖⁱ may be calculated and one of them as the centroid approximation tile xₖⁱ (cf. crosses with black frame in FIG. 1) may be randomly selected. The k centroid approximation tiles are then fed through θₑ followed by an aggregation layer θₐ for slide label prediction (cf. FIG. 1c). If there are no tiles assigned to a certain part of a slide, the input for that part is a zero-tensor of the same dimension as other input tiles. This integrated CNN model with kθₑ's and 1θₐ is optimized end-to-end by the slide loss. In out experiments, cross entropy loss after softmax was used for tumor vs non-tumor classification (cf. Section 4.1), while binary cross entropy after sigmoid activation was used for multi-label prediction (cf. Section 4.2). The k tile encoders θₑ share parameters during training.

Each slide can produce p^{k} samples in the form of {x₁ⁱ, . . . , xₖⁱ}. To avoid overfitting, only 10 samples may be constructed per training slide, collect samples of all slides, randomly shuffle them, and load a minibatch of size n for each iteration of training. In the meantime, random tiles may be sampled from the whole training dataset as a minibatch of size 2n for the centroid approximation learning (cf. FIG. 1d). The likelihood that the sampled tile features equal the corresponding centroids are maximized by MSE loss. The two losses are trained concurrently in each iteration.

3.4 Part Reassignment with Feature Attribution Estimation

As θₑ keeps being modified during training, the sampled tiles might no longer be good centroid approximations after certain iterations. Thus at the beginning of every epoch t, new global centroids {z₁, . . . , zₖ}^{t} may be calculated by averaging the new feature of each part of tiles assigned in the previous epoch t−1:zₖ^{t}=1/NΣₙ₌₁^{N}θₑ^{t}(x_{k,n}^{t-1}), then reassign tiles to the new centroids.

The part reassignment is based on the Euclidean distance between tile features and centroids. However, the assignment at epoch t should consider the feature attribution at t−1, especially when the feature length l is large. Otherwise, the important features learned from the previous epoch might be dominated by the other irrelevant ones in part formulation, which hinders the model learning convergence. The absolute value of the gradients may be used with respect to the slide loss of the kxl features to estimate their contribution. In every epoch, the attribution of each feature of all samples are averaged, and used for Euclidean distance calculation in a projected manifold for next epoch:

$$dist ( {θ}_{e} ( x ) , z ) = {( {\sum }_{i} {( {a}_{i} {{θ}_{e} ( x )}_{i} - {a}_{i} {z}_{i} )}^{2} )}^{0.5} \qquad ( 4 )$$

where aᵢ is the attribution of feature i.

In this manner, the part reassignment, the centroid approximation by the nearest tiles, as well as the MSE loss for pushing the feature of these tiles to their centroids, will weigh the important features more than others.

### 3.5 Architecture and Inference

Each θₑ is a ResNet34 whose last fc layer is replaced by a l₂ normalization, i.e. the feature vector after global average pooling is l₂ normalized. Also, the feature length is reduced from 512 to 1 by changing the output dimension of the last layer before global average pooling. θₐ is a one-layer fc layer. The model is implemented with PyTorch and trained on a single Volta V100 GPU.

During inference stage, for good prediction, all tiles of each slide may be used without any subsampling. According to the feature attribution and global centroids learned at training stage, the tiles are assigned to the k parts and the nearest 1 tile to each slide specific centroid is selected. The k tiles are fed through the model to output the slide prediction directly.

### 4. Experiments and Results

### 4.1 Clinical Grade Prostate and Basal Cell Carcinoma Classification

A novel MIL-RNN approach may be used to define and achieve clinical grade WSI classification of prostate cancer and basal cell carcinoma (BCC). The slides are labeled as positive if there are tumor regions on it, otherwise negative. This work provided with a strong evaluation baseline of WSI binary classification. The same training, validation and test slides. For BCC data, there are 6900 training slides, 1487 validation slides and 1575 test slides may be obtained. For prostate needle biopsies, there are 8521 training slides, 1827 validation slides and 1811 test slides. Each slide was tiled to patches of size 224×224 at 20× magnification, which corresponds to 0.5 microns per pixel. Note that the tumor purity varies hugely among the slides, and the tumor regions on many of them span only a few tiles.

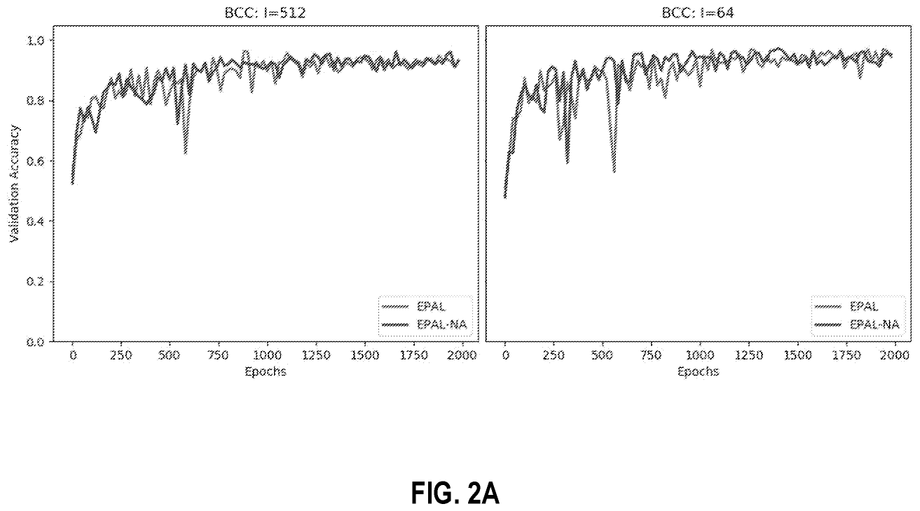

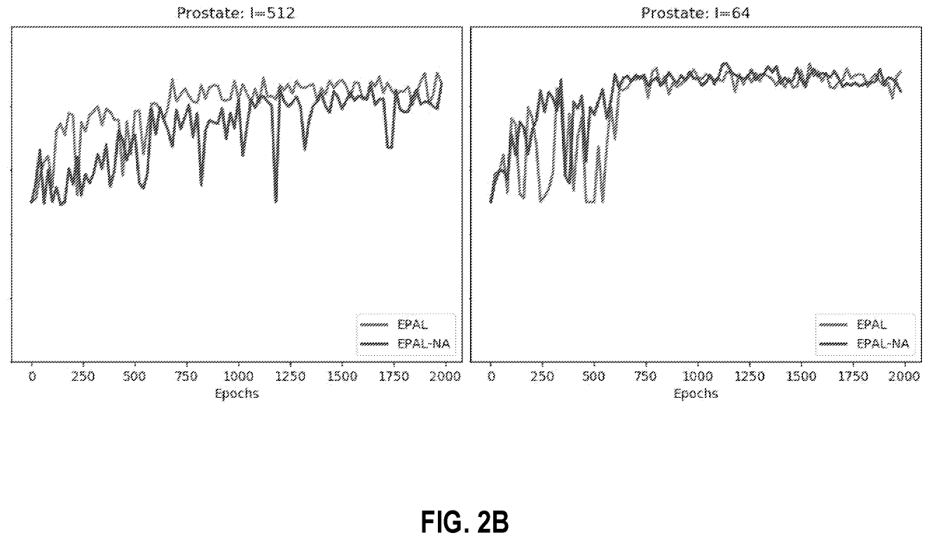

The effect on convergence of utilizing feature attribution (cf. Section 3.4) was evaluated. The EPL may be trained with and without feature attribution (EPL-NA) on the prostate and BCC datasets for 2000 epochs at learning rate 0.1 with a decay factor of 0.1 after every 600 epochs, which took about 300 hours. The best model and hyper-parameters were chosen based the performance on the validation dataset. A batch size n=32, k=8 clusters, and p=3 nearest tiles was used for centroid approximation. For convergence study, two feature lengths l=512 and l=64. Were compared. The convergence curves are shown in FIGS. 2A and 2B.

The convergence of model learning on BCC dataset was good with or without feature attribution (cf. FIG. 2A). For prostate, the optimization converged generally better with smaller l (cf. FIG. 2B). When l is large (512), EPL converged better than EPL-NA. These observations are consistent with the theory that the convergence should benefit from feature attribution when l is large (cf. Section 3.4), since there are more irrelevant noise features used in centroid approximation.

Since l=64 gave better validation results, this was used in the final test stage. Table 1 shows the area under ROC curve of different tasks. The left panel compared EPL to the MIL and MIL-RNN approaches. It shows that EPL can reach comparable results to MIL. MIL-RNN learns another aggregation RNN on top of the learned features and achieved slightly better AUC. It is noted that the other approach used quite strong tile-level supervision in their MIL training assuming that all tiles from non-cancer slides do not contain any tumor, i.e. Z=Y (cf. Section 3.1). EPL can also be concurrently trained with this extra loss, which would further increase the performance. However this assumption only holds for a small portion of the tasks that EPL can be applied to. Besides, adding labels to tiles violates EPL's nature of end-to-end training. Building an ensemble based on EPL, like adding an RNN on top of it, for optimal performance on certain tasks is beyond the scope of this paper. Nevertheless, the proposed method EPL achieved clinical grade performance for both prostate and basal cell carcinoma classification, with only 4 and 6 false negative slides (undetected cancer cases) out of the 1500+ test slides respectively. EPL-kl represents k=1. It lost the capability to combine information from diverse groups of tiles and resulted in much worse performance.

TABLE 1
Area under ROC curve for different classification tasks. Left: tumor
vs non-tumor classification of prostate cancer and basal cell carcinoma.
The results are from the MIL and MIL-RNN.
EPL has comparable clinical grade performance.
Right: multi-label classification of lung cancer architectural subtypes.
EPL-k1 represents the number of parts k = 1.
Diverse parts may be used for this multi-label classification task.

| | Prostate | BCC | Lepidic | Papillary | Solid | Micropapillary |
|---|---|---|---|---|---|---|
| MIL | 0.986 | 0.986 | — | — | — | — |
| MIL-RNN | 0.991 | 0.988 | — | — | — | — |
| EPL | 0.986 | 0.986 | 0.654 | 0.533 | 0.781 | 0.627 |
| EPL-NA | 0.984 | 0.987 | — | — | — | — |
| EPL-k1 | 0.734 | 0.930 | 0.585 | 0.518 | 0.648 | 0.530 |

Beyond classification, EPL provides rich information of WSIs that is crucial for high quality clinical decision support. For any task learned by EPL, the importance of various tile groups can be estimated by averaging the feature attributions of the group. FIG. 3 presents the part attributions side-by-side with the centroid approximation tiles used by EPL for prostate and BCC cancer classification. Each row corresponds to a part. Each column represents a test slide and the 8 tiles used for its classification. The heatmap bar represents the part attribution. 1's and 0's on top represents if it's cancer slide or not. A black square means that no tiles of this slide were assigned to this part.

The parts with high attribution for both tasks are basically the groups of cancer tiles (cf. FIG. 3 from above, left: row 7,8; right: row 1,7,8). For these parts, the negative slides either contain no tiles belonging to them (black squares) or have non-tumor tiles similar in appearance to tumor, which should be from the sparse intersections between groups on the manifold. EPL combined different morphological subtypes of tumors for the final prediction. These subtypes, along with other identified parts, have potential biological meaning and clinical relevance, which is worthy to be studied carefully as an extension of this work. These tiles can also be mapped back to their original positions on WSIs (cf. FIG. 5) for importance scoring of different regions over slide.

### 4.2 Multi-label Lung Cancer Architectural Subtyping

EPL's power of end-to-end learning of diverse features gives it potential for more complicated tasks of WSI assessment. As observed in prostate and BCC cancer classification (cf. Section 4.1), EPL has the potential to learn tumor subtypes, therefore the EPL was tested on a curated dataset of weakly-supervised lung cancer architectural subtype prediction. The dataset contains 599 lung cancer primary resection slides from patients with lung adenocarinoma. Each slide was labeled with a vector of binary entries each representing the existence of an architectural subtype of {lipidic, papillary, solid, micropapillary} as indicated in the surgical pathology report. Since it is a smaller dataset, a 5-fold cross-validation may be performed. The EPL may be trained on it for 1000 epochs with a learning rate 0.2 and a decay factor of 0.1 after every 400 epochs. Each epoch, all training slides and 100 tiles per slide may be used. Due to the small datasize, EPL began to overfit after 600 epochs, thus the best model and hyper-parameters may be selected based on the performance on the validation dataset before reaching the point of overfitting. A batch size n=32, k=12 clusters, feature lengths l=512, and p=1 nearest tiles may be used for centroid approximation.

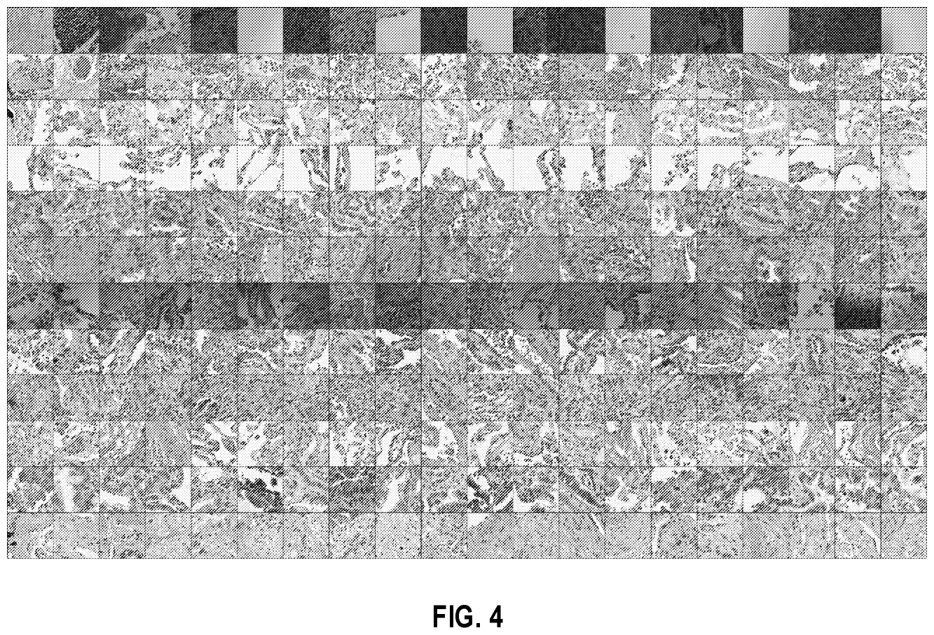

The area under ROC curve based on the prediction score of each subtype is shown in Table 1 right panel. For this weakly-supervised multi-label prediction task, MIL is not directly applicable, but rather requires to be combined with sophisticated tile sampling techniques. Among the 4 architectural subtypes, EPL predicted solid tumor the best, while learning the existence of papillary imposed hardness. When k=1, the capability of EPL degraded due to the loss of feature diversity. It was found that the groups learned by EPL were formed such that some of them directly corresponded to the four subtypes used for training quite well (cf. FIG. 4 row 6,9,10,11). Each row in FIG. 4 is the 20 nearest tiles of the whole dataset to the learned global centroids. Note that although not used in the slide label, the clinically relevant acinar subtype was enriched in part 7 (cf. FIG. 4 row 7). Given these observations, it may be surmised that gathering more data would help preventing the overfitting and achieving better results.

### 5. Discussion

As a general weakly-supervised WSI prediction algorithm, EPL was built upon the least assumptions comparing to MTh approaches, and thus theoretically applicable to a plethora of tasks. This, however, implies that EPL would require very large datasets to be most successful. To achieve better accuracy on smaller datasets, EPL can be easily combined with limited annotations or constraints. For example, EPL applied to the lung subtyping task described in this paper can be concurrently trained with annotations on a small number of tiles. Another extension of EPL is its application on survival regression. Modeling WSIs as clusters of tiles is uniquely powerful for survival analysis. Implementing EPL on survival analysis differs from them by not only learning tile groups, but also forming the groups based on slide level supervision. With the simplicity and power of its end-to-end structure, it is suggested that EPL can be the backbone or framework, based on which, many innovative and efficient models can be developed for WSI assessment.

### B. Systems and Methods for Classifying Biomedical Image Using Machine Learning Models

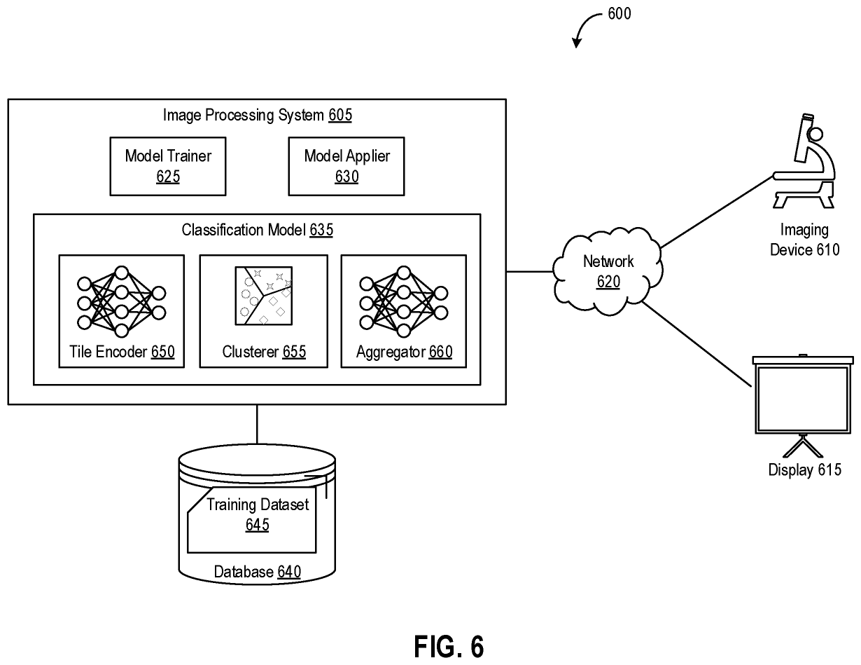

Referring now to FIG. 6, depicted is a block diagram depicting a system 600 for classifying biomedical images using machine learning models. In overview, the system 600 may include at least one image processing system 605, at least one imaging device 610, and at least one display 615, among others, communicatively coupled via at least one network 620. The image processing system 605 may include at least one model trainer 625, at least one model applier 630, at least one classification model 635, and at least one database 640, among others. The database 640 may store, maintain, or otherwise include at least one training dataset 645. The classification model 635 may include at least one tile encoder 650, at least one clusterer 655, and at least one tile aggregator 660, among others. Each of the components in the system 600 as detailed herein may be implemented using hardware (e.g., one or more processors coupled with memory) or a combination of hardware and software as detailed herein in Section C. Each of the components in the system 600 may implement or execute the functionalities detailed herein, such as those described in Section A.

In further detail, the image processing system 605 itself and the components therein, such as the model trainer 625, the model applier 630, and the classification model 635 may have a training mode and a runtime mode (sometimes herein referred to as an evaluation or inference mode). Under the training mode, the image processing system 605 may invoke the model trainer 625 to train the classification model 635 using the training dataset 645. Under the runtime, the classification model 635 may invoke the model applier 630 to apply the classification model 635 to new incoming biomedical images from the imaging device 610.

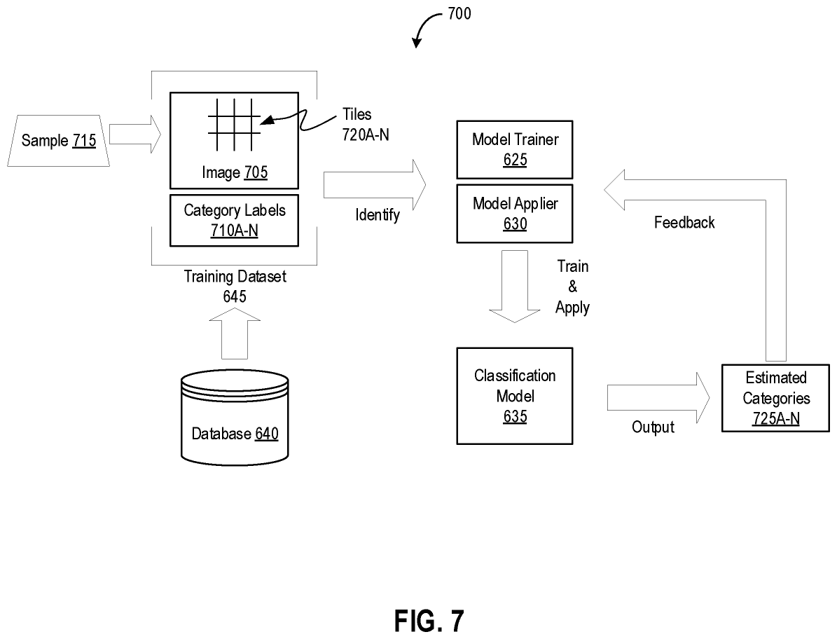

Referring now to FIG. 7, depicted is a block diagram of a training process 700 in the system 600 for classifying biomedical images. The process 700 may correspond to or include at least a subset of operations performed by the image processing system 605 under the training mode. Under the process 700, the model trainer 625 executing on the image processing system 605 may initialize, train, or otherwise establish the classification model 635 using the training dataset 645. In initializing, the model trainer 625 may assign values (e.g., random values) to the weights and parameters of the classification model 635. To train the classification model 635, the model trainer 625 may access the database 640 to fetch, retrieve, or identify the training dataset 645. The training dataset 645 may be stored and maintained on the database 640 using at least one data structure (e.g., an array, a matrix, a heap, a list, a tree, or a data object). With the identification, the model trainer 625 may train the classification model 635 using the training dataset 640. The training of the classification model 635 may be in accordance with weakly supervised learning.

The training dataset 645 may include one or more examples. Each example in the training dataset 645 may identify or include at least one image 705 and one or more category labels 710A-N (herein after generally referred to as category labels 710). Each example may be associated with at least one sample 715. The sample 715 may be a tissue section taken or obtained from a subject (e.g., a human, animal, or flora). For example, the tissue section for the sample 715 may include a muscle tissue, a connective tissue, an epithelial tissue, nervous tissue, or an organ tissue, in the case of a human or animal subject. The sample 715 may have or include one or more objects with one or more conditions. For instance, the tissue section for the sample 715 may contain various cell subtypes corresponding to different conditions, such as carcinoma, benign epithelial, background, stroma, necrotic, and adipose, among others.

In the training dataset 645, the image 705 may be acquired, derived, or otherwise may be of the sample 715. The image 705 itself may be acquired in accordance with microscopy techniques or a histopathological image preparer, such as using an optical microscope, a confocal microscope, a fluorescence microscope, a phosphorescence microscope, an electron microscope, among others. The image 705 may be, for example, a histological section with a hematoxylin and eosin (H&E) stain, immunostaining, hemosiderin stain, a Sudan stain, a Schiff stain, a Congo red stain, a Gram stain, a Ziehl-Neelsen stain, a Auramine-rhodamine stain, a trichrome stain, a Silver stain, and Wright's Stain, among others.

The image 705 may include one or more regions of interest (ROIs). Each ROI may correspond to areas, sections, or boundaries within the sample image 705 that contain, encompass, or include conditions (e.g., features or objects within the image). For example, the sample image 705 may be a whole slide image (WSI) for digital pathology of a tissue section in the sample 715, and the ROIs may correspond to areas with cancerous or lesion cells. In some embodiments, the ROIs of the sample image 705 may correspond to different conditions. Each condition may define or specify a category for the ROI. For example, when the image 705 is a WSI of the sample tissue, the conditions may correspond to various histopathological characteristics, such as carcinoma tissue, benign epithelial tissue, stroma tissue, necrotic tissue, and adipose tissue, among others.

For the associated image 705, each category label 710 may define, specify, or otherwise identify a presence or a lack of a corresponding condition in the sample 715 from which the image 705 is derived. By extension, the category label 710 may also identify the presence or the absence of ROIs for the corresponding condition in the image 705. The condition may include, for example, as carcinoma tissue, benign epithelial tissue, stroma tissue, necrotic tissue, and adipose tissue, among others. Each example of the training dataset 645 may include multiple category labels 710 corresponding to multiple conditions. For example, the first category label 710A may indicate whether the image 705 has any ROIs corresponding to carcinoma cells in the depicted sample 715, whereas another category label 710B may indicate whether the image 705 has any ROIs corresponding to necrotic tissue. The category labels 710 may be manually generated or inputted by a pathology examining the image 705 or the sample 715. To facilitate weakly-supervised learning, the category label 710 may lack identification of specification locations of the ROI indicating presence or absence of the condition in the image 705. The category label 710 instead may indicate that the condition is present somewhere within the image 705.

In training the classification model 635, the model applier 630 executing on the image processing system 605 may identify or generate the set of tiles 720A-N (hereinafter generally referred to as tiles 720) from the image 705 from each example of the training dataset 645. Each tile 720 may correspond to a portion of the image 705 in the example. In some embodiments, the set of tiles 720 may be defined in the example of the training dataset 645. The model applier 630 may identify the set of tiles 720 from the image 705 in accordance with the definition in the example. In some embodiments, the model applier 630 may partition or divide the image 705 into the set of tiles 720. The set of tiles 720 may be disjoined or may be overlap with one another. In some embodiments, the model applier 630 may generate the set of tiles 720 from the image 705 with an overlap in accordance with a set ratio. The ratio may range from 10% to 90% overlap between pairs of adjacent tiles 720.

In some embodiments, the model applier 630 may identify or detect one or more areas within the image 705 from each example of the training dataset 645. In some embodiments, the areas may correspond to a positive space within the image 705. The identification of the positive space may be based on a visual characteristic of the pixels in the image 705. For example, the positive space may correspond to areas of the image 705 that is neither white nor null as indicated by the red, green, blue (RGB) values of the pixels in the areas. With the identification, the model applier 630 may generate the set of tiles 720 using the areas corresponding to positive space within the image 705. Conversely, in some embodiments, the areas may correspond to a negative space within the image 705. The identification of the negative space may be based on a visual characteristic of the pixels in the image 705. For example, the positive space may correspond to areas of the image 705 that is white nor null as indicated by the RGB values of the pixels in the areas. The model applier 630 may remove the areas corresponding to the negative space from the image 705. Using the remaining portion of the image 705, the model applier 630 may generate the set of tiles 720.

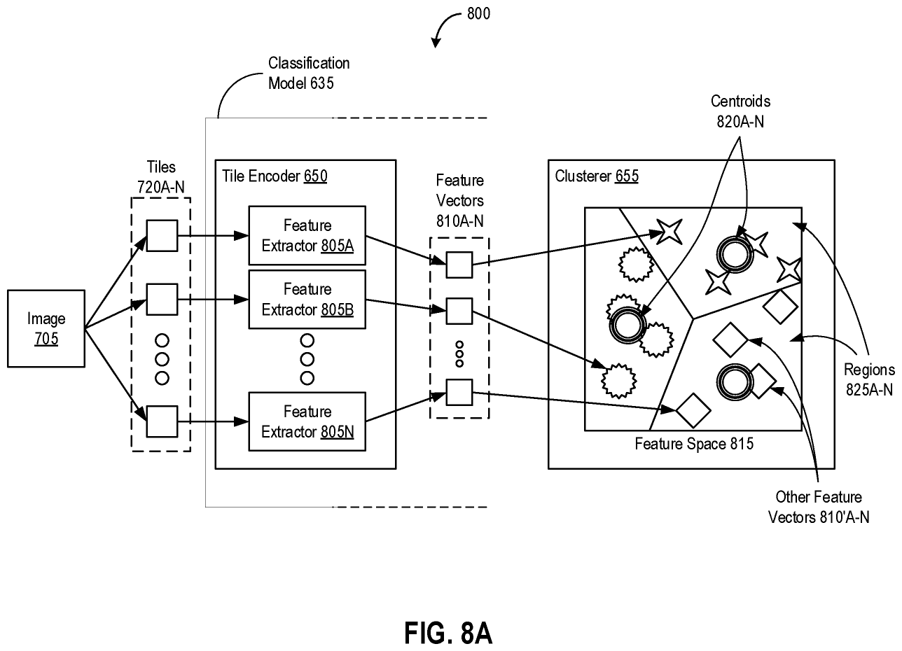

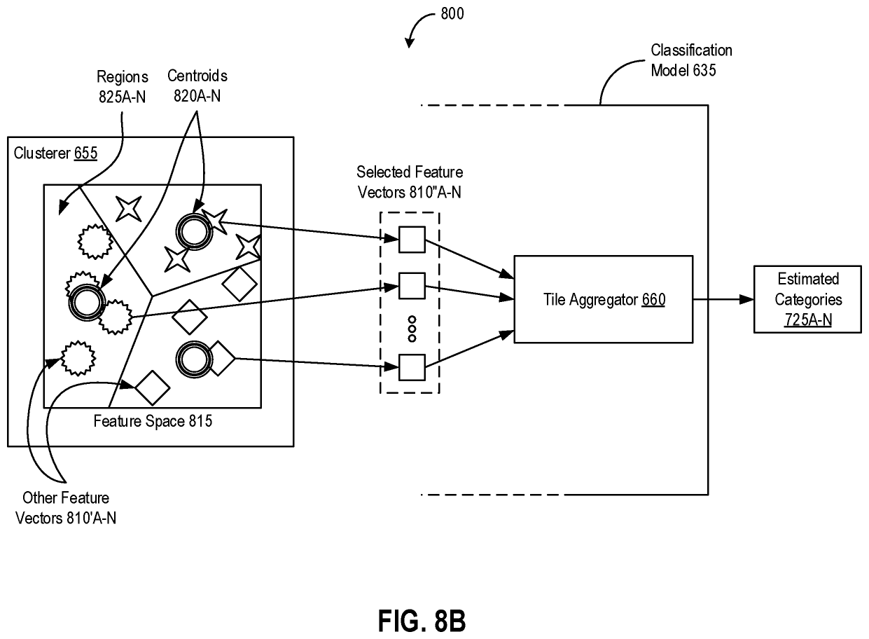

With the identification, the model applier 630 may apply the set of tiles 720 from the image 705 of each example in the training dataset 645 to the classification model 635. The classification model 635 may include or have a set of weights (sometimes herein referred to as parameters, kernels, or filters) and a set of centroids to process inputs and produce outputs. The set of weights may be arranged or defined in the classification model 635, for example, in accordance with a convolutional neural network (CNN) architecture in the tile encoder 650 and the tile aggregator 660. The set of centroids may be arranged or defined in the classification model 635, for example, in a feature space defined by the clusterer 655. When initialized, both the weights and the centroids may be assigned to set values (e.g., random values). Details of the architecture and functionality of the tile encoder 650, the clusterer 655, and the tile aggregator 660 are described herein below in conjunction with FIGS. 8A-10.

In applying, the model applier 630 may provide or feed the tiles 720 of the image 705 from each example of the training dataset 645 as the input to the classification model 635. Upon feeding, the model applier 630 may process the input tiles 720 in accordance with the set of weights and centroids of the classification model 635 to generate at least one output. The output may include a set of estimated categories 725A-N (hereinafter generally referred to estimated categories 725). Each estimated category 725 may specify, define, or identify a corresponding condition in at least one of the tiles 720 of the image 705. In some embodiments, the output may include a set of confidence scores for each estimated category 725. The confidence score may define or indicate a likelihood that at least one of the tiles 720 in the image 705 includes a feature for the condition corresponding to the estimated category 725. In some embodiments, the output may include a subset of tiles 720 selected from the overall set and a set of confidence scores (sometimes herein referred to as importance scores) for each estimated categories 725. The subset of tiles 720 may be selected based on the condition for the corresponding estimated category 725. The set of confidence scores may be associated with the corresponding subset of tiles 720. Each confidence score may define or indicate a likelihood that the corresponding tile 720 includes a feature for the condition corresponding to the estimated category 725.

Referring now to FIGS. 8A and 8B, depicted are block diagrams of an architecture 800 for the classification model 635 in the system 600 for classifying biomedical images. Starting from FIG. 8A, under the architecture 800 for the classification model 635, the tile encoder 650 may include a bank or a set of feature extractors 805A-N (hereinafter generally referred to as feature extractors 805). At least some of the set of weights of the classification model 635 may configured, arrayed, or otherwise arranged across the tile encoder 650, including the set of feature extractors 805 therein. The inputs to the classification model 635 may be provided or fed by the model applier 630 as the inputs to the tile encoder 650. The inputs may include the set of tiles 720 from the images 705 from one or more examples in the training dataset 645. Each input may be provided or fed as the input to a corresponding feature extractor 805.

The number of feature extractors 805 included in the tile encoder 705 may be dependent on the number of tiles 720. In some embodiments, the number of feature extractors 805 in the tile encoder 705 may correspond to the total number of tiles 720 inputted into the tile encoder 705 from one image 705. For example, for each tile 720 from one image 705 inputted into the classification model 635, the tile encoder 705 may have a corresponding feature extractor 805 to process the input tile 720. In some embodiments, the number of feature extractors 805 in the tile encoder 650 may correspond to the set of tiles 720 from multiple images 705 from multiple examples of the training dataset 645. In some embodiments, the number of feature extractors 805 may correspond to the number of tiles 720 inputted at a given instance. In some embodiments, the number of feature extractors 805 may correspond to the number of tiles 720 inputted at an epoch of training.

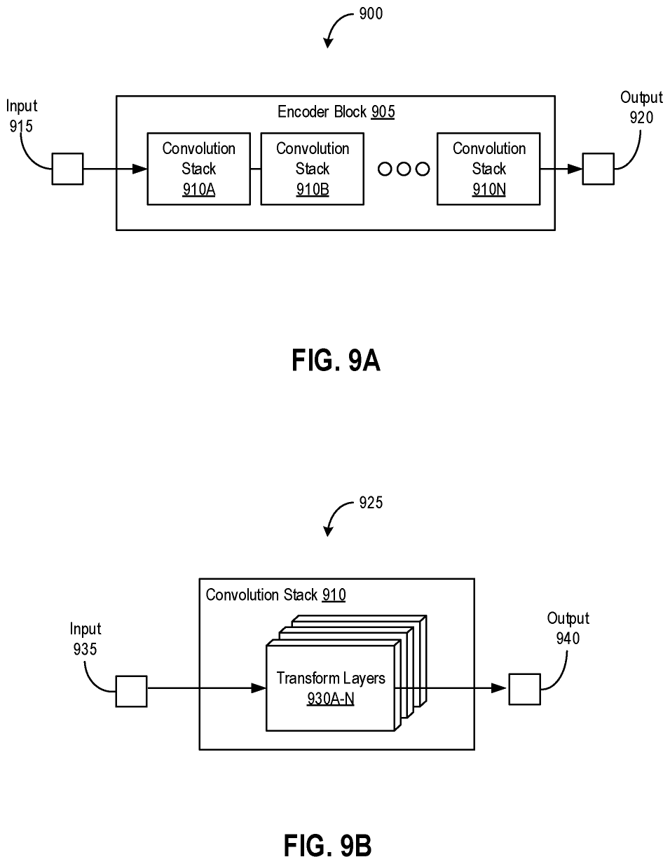

In the tile encoder 650, each feature extractor 805 may receive, retrieve, or otherwise identify the input tile 720 from the image 705. Upon receipt, the feature extractor 805 may process the tile 720 in accordance with the set of weights. The set of weights in the feature extractor 805 may be arranged, for example, according to a convolutional neural network (CNN). In some embodiments, the set of weights may be shared amongst the feature extractor 805. For example, the values and interconnections of the weights within the feature extractor 805 may be the same throughout the feature extractors 805 in the classification model 635. In some embodiments, the set of weights may be not shared among the feature extractors 805. For instance, the values or the interconnections of the weights in one feature extractor 805 may differ or may be independent of the values or the interconnections of the weights in other feature extractors 805. The feature extractor 805 may be implemented using the architectures detailed herein in conjunction with FIGS. 9A and 9B.

From processing the tile 720 using the weights, the feature extractor 805 may produce or generate at least one feature vector 810A-N (hereinafter generally referred to as feature vector 810). The feature vector 810 may be a lower dimensional representation of the input tile 720. For example, the feature vector 810 may be an embedding, encoding, or a representation of latent features in the input tile 720. The feature vector 810 may be n-dimensional and may include n values along each dimension. The values in each dimension may likewise be a representation of latent features from the tile 720. The set of feature vectors 810 outputted by the set of feature extractors 805 in the tile encoder 650 may be provided or fed forward as inputs to the clusterer 655.

The clusterer 655 may retrieve, receive, or otherwise identify the set of feature vectors 810 outputted by the set of feature extractors 805 of the tile encoder 650 as input to define or map against at least one feature space 815. The clusterer 655 may include or define the feature space 815. The feature space 815 may be an n-dimensional space in which each feature space 815 can be defined. The feature space 815 may define or otherwise include a set of centroids 820A-N (hereinafter generally referred to as centroids 820). Each centroid 820 may correspond to a data point in the n-dimensional feature space 815. Upon initialization during the training process, the set of centroids 820 in the clusterer 655 may be assigned a set value (e.g., a random value). The set of centroids 820 may be used to delineate, demarcate, or otherwise define a corresponding set of regions 825A-N (hereinafter generally referred to as regions 825) within the feature space 815. Each region 825 may correspond to a portion of the feature space 815. In some embodiments, each region 825 may correspond to the portion of the feature space 815 based on a distance about the associated centroid 825 in the feature space 815. The distance may be, for example, proximity in terms of Euclidean distance or L-norm distance, among others, to the centroid 825 defining the respective region 825. Each region 825 and corresponding centroid 820 may correspond to a part or one of the latent parameters correlated with a condition to which the image 705 is to be categorized.

Upon receipt, the clusterer 655 may assign or map each feature vector 810 to the feature space 815. To assign, the clusterer 655 may identify values along each dimension of the feature vector 810. Based on the values along the dimensions, the clusterer 655 may identify a point within the feature space 815 to map the feature vector 810 against. For example, the feature vectors 810 produced by the tile encoder 650 may be n-dimensional and each feature vector 810 may be mapped as a data point using the values along each of the n dimensions. within the feature space 815. When assigning feature vectors 810 generated from the tiles 702 of one image 705, the clusterer 655 may have already assigned or mapped other feature vectors 810′A-N (hereinafter generally referred to as other feature vectors 810′) from the tile 702 of other images 705 in the training dataset 645.

With the mapping of each feature vector 810 within the feature space 815, the clusterer 655 may also determine or identify the region 825 to which to assign the feature vector 810. For each feature vector 810, the clusterer 655 may calculate or determine a distance between the point corresponding to the feature vector 810 and each centroid 820 within the feature space 815. The distance may be determined in accordance with Euclidean distance or L-norm distance, among others. Based on the distances to the centroids 820 within the feature space 815, the clusterer 655 may assign the feature vector 810 to one of the regions 825. For example, the clusterer 655 may assign the feature vector 810 to the region 825 associated with the most proximate centroid 820 within the feature space 815. In some embodiments, the clusterer 655 may identify the region 825 to which to assign the feature vector 810 based on the values along the dimensions of the feature vector 810. As discussed above, the clusterer 655 may have already partitioned the feature space 815 into the set of regions 825 based on the associated set of centroids 820. The clusterer 655 may compare the values along the dimensions of the feature vector 810 with the values of the feature space 815 associated with the set of regions 825. Based on the comparison, the clusterer 655 may assign the feature vector 810 to the region 825 in which the values along the dimensions reside. The other feature vectors 810′ may have been assigned to the regions 825 of the feature space 815 in a similar manner.

Moving onto FIG. 8B, the clusterer 655 may identify or select a subset of feature vectors 810″A-N (hereinafter generally referred to as subset of feature vectors 810″) based on the centroids 820 or regions 825 in the feature space 815. The selected subset of feature vectors 810″ may be associated with one image 705 of one example in the training dataset 745. By extension, the selected subset of features 810″ may correspond to a subset of tiles 720 from the image 705. The selected subset of feature vector 810″ may include at least one feature vectors 810 from each of the regions 825 in the feature space 815.

To identify, the clusterer 655 may identify the superset of feature vectors 810 within the feature space 815 generated from the tiles 720 for each image 705. From the superset of feature vectors 810 associated with the image 705, the clusterer 655 may select or identify the feature vectors 810 in each region 825 of the feature space 815. From the feature vectors 810 in each region 825, the clusterer 655 may select or identify one feature vector 810″ for the subset based on a distance of the feature vector 810″ to the centroid 820 used to define the respective region 825. The distance may be in terms of Euclidean distance or L-norm distance, among others. The feature vector 810″ selected may correspond to the feature vector 810 closest to the centroid 820 in the associated region 825. In some embodiments, the clusterer 655 may identify the subset of tiles 720 that correspond to the subset of feature vectors 810″ selected from the superset of feature vector 810. The selected subset of feature vectors 810″ may be provided or fed forward by the model applier 630 as an input to the aggregator 650. The selected subset of tiles 720 may be provided as an output of the overall classification model 635.

The tile aggregator 660 may retrieve, receive, or otherwise identify the subset of feature vectors 810″ selected by the clusterer 855. In some embodiments, the tile aggregator 660 may concatenate, conjoin, or otherwise combine the subset of feature vectors 810″ upon receipt. The tile aggregator 660 may process the input subset of feature vectors 810″ in accordance with the set of weights defined at the tile aggregator 660. The set of weights in the tile aggregator 660 may be arranged, for example, according to a convolutional neural network (CNN). The feature tile aggregator 660 may be implemented using the architectures detailed herein in conjunction with FIGS. 9A and 9B. In some embodiments, the tile aggregator 660 may process the combined subset of feature vectors 810″ (e.g., a concatenation of feature vectors 810″) using the set of weights.

From processing the feature vectors 810″, the tile aggregator 660 may produce or generate at least output. The output may include the output of the overall classification model 635, as discussed above. The tile aggregator 660 may generate or determine the set of estimated categories 725 using the selected feature vectors 810″. Each estimated category 725 may identify a corresponding condition in at least one of the tiles 720 in the image 705. In some embodiments, the tile aggregator 660 may determine a confidence score for each of the estimated categories 725. The confidence score may define or indicate a likelihood that at least one of the tiles 720 in the image 705 includes a feature for the condition corresponding to the estimated category 725. In some embodiments, the tile aggregator 660 may sort or rank the estimated categories 725 by the corresponding confidence scores.

In some embodiments, the tile aggregator 660 may determine or generate an association between each tile 720 and one of the estimated categories 725. In generating, the tile aggregator 660 may determine or identify a component score for each feature vector 810″ to each estimated category 725. The component score may measure or indicate a degree of impact to which the feature vector 810″ contributed to the overall confidence score for the corresponding estimated category 725. Based on the component scores, the tile aggregator 660 may identify or select one feature vector 810″ for each estimated category 725. For instance, the feature vector 810″ with the highest component score may be selected for the estimated category 725. Upon identification, the tile aggregator 660 may select or identify the tile 720 corresponding to the feature vector 810″. The tile aggregator 660 may use the component score of the feature vector 810″ as the confidence score for the associated tile 720 in relation to the estimated category 725.

Referring now to FIG. 9A, depicted is a block diagram of an architecture 900 of an encoder block 905 used to implement the classification model 635 in the system for classifying biomedical images. The encoder block 905 may be used to implement the individual feature extractors 805 as well as the tile aggregator 660 in the classification model 635. For example, each feature extractor 805 and the tile aggregator 660 may be an instance of the encoder block 905. Under the architecture 900, the encoder block 905 may include one or more convolution stacks 910A-N (hereinafter generally referred to as convolution stacks 910). The encoder block 910 may also include at least one input 915 and at least one output 920. The input 915 and the output 920 may be related via the set of weights defined in the convolution stacks 910. When used to implement the feature extractor 805, the input 915 of the encoder block 905 may correspond to or include the tile 720 from the image 705 and the output 920 may correspond or include the feature vector 810. When used to implement the tile aggregator 660, the input 915 of the encoder block 905 may correspond to or include the subset of feature vectors 810″ and the output may include the set of estimated categories 725 and other outputs described above. Each convolution stack 910 may define or include the weights the encoder block 905. The set of convolution stacks 910 can be arranged in series (e.g., as depicted) or parallel configuration, or in any combination. In a series configuration, the input of one convolution stacks 910 may include the output of the previous convolution stacks 910 (e.g., as depicted). In parallel configuration, the input of one convolution stacks 910 may include the input of the entire encoder block 905. Details regarding the architecture of the convolution stack 910 are provided herein below in conjunction with FIG. 9B.

Referring now to FIG. 9B, depicted is a block diagram of an architecture 925 of the convolution stack 910 of the encoder block 905 used to implement the classification model 635 in the system 600 for classifying biomedical images. Under the architecture 925, the convolution stack 910 may include one or more transform layers 930A-N (hereinafter generally referred to as transform layers 930). The convolution stack 910 also include at least one input 935 and at least one output feature map 490. The input 935 and the output 940 may be related via the set of weights defined in the transform layers 930 of the convolution stack 910. The set of transform layers 930 can be arranged in series, with an output of one transform layer 930 fed as an input to a succeeding transform layer 930. Each transform layer 930 may have a non-linear input-to-output characteristic. The transform layer 930 may comprise a convolutional layer, a normalization layer, and an activation layer (e.g., a rectified linear unit (ReLU), softmax function, or a sigmoid function), among others. In some embodiments, the set of transform layers 930 may be a convolutional neural network (CNN). For example, the convolutional layer, the normalization layer, and the activation layer may be arranged in accordance with CNN. The activation layer may be a softmax function for binary classifications and may be a sigmoid function for non-binary classifications.

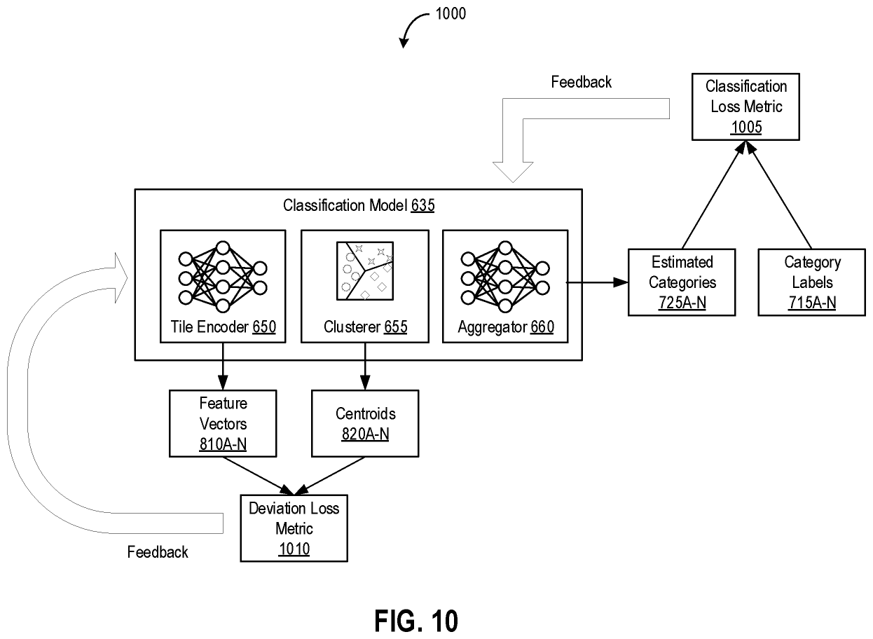

FIG. 10 is a block diagram of an updating process 1000 in the system 600 for classifying biomedical images using machine learning models. The process 1000 may correspond to or include at least a subset of operations performed by the image processing system 605 under the training mode. Under the process 1000, the model trainer 625 may retrieve, obtain, or otherwise identify the output generated by the classification model 635 from the application of the image 705. The output may include, for example, the one or more estimated categories 725. The model trainer 625 may also identify the example of the training dataset 645 that includes the image 705 from which the tiles 720 were inputted into the classification model 635. The model trainer 625 may retrieve or identify the category labels 715 from the same example in the training dataset 645.

With the identification, the model trainer 625 may compare the estimated categories 725 generated by the classification model 635 and the category labels 715 in the example of the training dataset 645. Based on the comparison, the model trainer 625 may determine whether the category labels 715 correspond to or match the estimated categories 725. In some embodiments, the model trainer 625 may determine a number of the estimated categories 725 that match or do not match the category labels 715. The model trainer 625 may traverse through the set of estimated categories 725 in performing the comparisons. If the estimated category 725 differs from all of the category labels 715 in the example, the model trainer 625 may determine that the estimated category 725 does not match any of the category labels 715. Furthermore, the model trainer 625 may also increment the number of non-matching estimated categories 725. Conversely, if the estimated category 725 is the same as at least one of the category labels 715, the model trainer 625 may determine that the estimated category 725 matches the corresponding category label 715. In addition, the model trainer 625 may also increment the number of matching estimated categories match. The comparison may be repeated over all of the estimated categories 725.

Based on comparison between the estimated categories 725 and the category labels 715, the model trainer 625 may calculate, generate, or otherwise determine at least one classification loss metric 1005. The classification loss metric 1005 may indicate a degree of deviation of the estimated categories 725 outputted by the classification model 635 from the expected category labels 715 as identified in the example of the training dataset 645. In some embodiments, the model trainer 625 may determine the classification loss metric 1005 based on the number of matching estimated categories 725 or the number of non-matching estimated categories 725, or both. The classification loss metric 1005 may be calculated in accordance with any number of loss functions, such as a norm loss (e.g., L1 or L2), mean squared error (MSE), a quadratic loss, a cross-entropy loss, and a Huber loss, among others. In general, the higher the classification loss metric 1005, the more the output may have deviated from the expected result of the input. Conversely, the lower the classification loss metric 1005, the lower the output may have deviated from the expected result.

Using the classification loss metric 1005, the model trainer 625 modify, set, or otherwise update the set of weights across the classification model 635, such as the weights in the tile encoder 650 and the tile aggregator 660. The weights of the tile encoder 650 and the tile aggregator 660 may be updated using the classification loss metric 1005 in the same feedback, for example, in an end-to-end manner as described in Section A. In some embodiments, the model trainer 625 may use the classification loss metric 1005 to update the set of centroids 820 in the clusterer 655. The updating of weights may be in accordance with an optimization function (or an objective function) for the tile encoder 650 and the tile aggregator 660 in the classification model 635. In The optimization function may define one or more rates or parameters at which the weights of the classification model 635 are to be updated. The updating of the parameters in the classification model 635 may be repeated until convergence.

In addition, the model trainer 625 may calculate, generate, or otherwise determine at least one deviation loss metric 1010 based on comparison between the set of feature vectors 810 and the centroids 820 within the feature space 815. The deviation loss metric 1010 may indicate a degree of dispersion, spread, or distance between the centroid 820 and each of the feature vectors 810 assigned to the region 825. The determination of the deviation loss metric 1010 may be based on a comparison between each centroid 820 and each of the feature vectors 810 assigned to the region 825 within the feature space 815. In comparing, the model trainer 625 may calculate or determine a distance between the centroid 820 and each feature vector 810 in the region 825 associated with the centroid 820. The distance may be in accordance with Euclidean distance or L-norm distance, among others. Using the distances over all the centroids 820, the model trainer 625 may determine the deviation loss metric 1010. The deviation loss metric 1010 may be calculated in accordance with any number of loss functions, such as a norm loss (e.g., L1 or L2), mean squared error (MSE), a quadratic loss, a cross-entropy loss, and a Huber loss, among others. In general, the higher the deviation loss metric 1010, the more the spread or distance between each centroid 820 and assigned feature vectors 810. Conversely, the lower the deviation loss metric 1010, the lower the spread or distance between each centroid 820 and the assigned feature vectors 810.

Using the deviation loss metric 1010, the model trainer 625 modify, set, or otherwise update the set of weights in the classification model 635, such as the weights in the individual feature extractors 805 in the tile encoder 650. In some embodiments, the model trainer 625 may use the deviation loss metric 1010 to update the set of centroids 820 in the clusterer 655. The updating of weights may be in accordance with an optimization function (or an objective function) for the tile encoder 650 in the classification model 635. In The optimization function may define one or more rates or parameters at which the weights of the classification model 635 are to be updated. The updating of the parameters and the centroids 820 in the classification model 635 may be repeated until convergence.

With the updating of the weights in the tile encoder 650 and the tile aggregator 660, the model trainer 625 may invoke the model applier 630 to reapply the tiles 720 of the image 705 from each example of the training dataset 645. The model applier 630 may repeat the process of the applying the classification model 635 as described herein above to commence another training epoch. In applying, the model applier 630 may provide or feed the tiles 720 of the image 705 into the tile encoder 650 of the classification model 635. The model applier 630 may process the tiles 720 in accordance with the updated weights of the feature extractors 805 of the tile encoder 650 to generate a new set of feature vectors 810. The new feature vectors 810 may contain new values different from the previous feature vectors 810, as updated weights in the feature extractors 805 are used to generate the new feature vectors 810. Upon generation, the model applier 630 may invoke the clusterer 655 to map the newly generated set of feature vectors 810 to the feature space 815. Because the feature vectors 810 contain new values, the new set of feature vectors 810 may be mapped to different points within the feature space 815 relative to the prior set of feature vectors 810.

Based on the assignment of new feature vectors 810 in the feature space 815, the model trainer 625 may change, set, or otherwise update the set of centroids 820 of the clusterer 655 within the feature space 815. To update, the model trainer 625 may identify the feature vectors 810 previously assigned to each region 825 from the previous application of the tiles 720. For each region 825, the model trainer 625 may identify the values of each feature vector 810 within the feature space 815. Using the values, the model trainer 625 may determine the new values for each centroid 820 in the feature space 815. In some embodiments, the model trainer 625 may determine the centroid 820 based on a combination (e.g., mean) of the values of the feature vectors 810 assigned to the region 825. Once determined, the model trainer 625 may update the each centroid 820 to the respective new values within the feature space 815. With the re-assignment of the centroids 820, the training and application process may be repeated as described above upon convergence.

Upon convergence, the model trainer 625 may store and maintain the set of weights in the tile encoder 650 and the tile aggregator 660 and the set of centroids 820 of the clusterer 655. The convergence may correspond to a change in the values of weights in the tile encoder 650 and the tile aggregator 660 of less than a threshold value. The convergence may also correspond to a change in the values of the centroids 820 of less than some threshold value. The set of weights in the tile encoder 650 and the tile aggregator 660 and the set of centroids 820 may be stored and maintained using one or more data structures, such as an array, a matrix, a heap, a list, a tree, or a data object, among others. In some embodiments, the model trainer 625 may store and maintain the weights and the centroids 820 on the database 640.

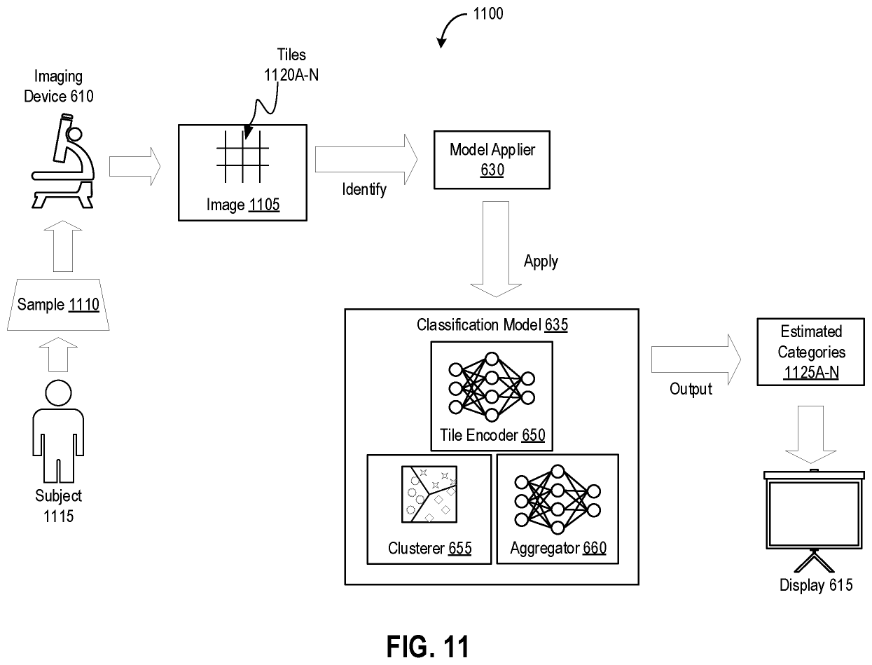

FIG. 11 is a block diagram of an inference process 1100 in the system 600 for classifying biomedical images using machine learning models. The process 1100 may correspond to or include operations performed by the image processing system 605 under evaluation mode. The operations performed under evaluation mode may overlap or may be similar to the operations performed under training mode as discussed above. Under the process 1100, the imaging device 610 may scan, obtain, or otherwise acquire at least one image 1105 of at least one sample 1110 from a subject 1115. The image 1105 may be similar to the image 705 described above, but may be newly acquired from the imaging device 610. For instance, the image 1105 may be a histological section corresponding the sample 1110 with a hematoxylin and eosin (H&E) stain acquired via an optical microscope. The sample 1110 may be a tissue section with various cell subtypes corresponding to different conditions, such as carcinoma, benign epithelial, background, stroma, necrotic, and adipose, among others. Upon acquisition, the imaging device 610 may send, transmit, or otherwise provide the acquired image 1105 to the imaging processing system 605.

The model applier 630 may in turn retrieve, receive, or otherwise identify the image 1105 from the imaging device 610. The model applier 630 may process the image 1105 in a similar manner as detailed above with respect to the image 705. Upon receipt, the model applier 630 may generate the set of tiles 1120A-N (hereinafter generally referred to as tiles 1120) from the image 1105. The tiles 1120 may be generated from areas within the image 1105 determined to correspond to positive space. With the generation, the model applier 630 may apply the tiles 1120 from the image 1105 to the classification model 635. In applying, the model applier 630 may provide or feed the tiles 1120 of the image 1105 as input into the classification model 635. Upon feeding, the model applier 630 may process the input tiles 1120 with the set of weights and centroids of the classification model 635 to generate at least one output. The processing may be similar as described above in relation to the input tiles 720, and the output may be similar as described above.

Upon generation, the model applier 630 may store and maintain an association between the image 1105 and the output from the classification model 635. The association may be stored on the database 640 using one or more data structures, such as an array, a matrix, a heap, a list, a tree, or a data object, among others. The output by the classification model 635 may include a set of estimated categories 1125A-N (hereinafter generally referred to estimated categories 1125). Each estimated category 1125 may specify, define, or identify a corresponding condition in at least one of the tiles 1120 of the image 1105. In some embodiments, the output may include a set of confidence scores for each estimated category 1125. In some embodiments, the output may include a subset of tiles 1120 selected from the overall set and a set of confidence scores (sometimes herein referred to as importance scores) for each estimated categories 1125. The subset of tiles 1120 may be selected based on the condition for the corresponding estimated category 1125. The set of confidence scores may be associated with the corresponding subset of tiles 1120.

In addition, the model applier 630 may send, transmit, or otherwise provide the output from the classification model 635 to the display 615 for presentation. The display 615 may be part of the image processing system 605 or may be of another computing device. In some embodiments, the model applier 630 may provide the association to the display 615. With the receipt, the display 615 may render or present information associated with the output or the association. In some embodiments, the display 615 may present the image 1105 (or tiles 1120) along with the estimated categories 1125 and the confidence scores. For example, the display 615 may render a graphical user interface (GUI) with the image 1105, the estimated categories 1125, and the associated confidence scores. In some embodiments, the display 615 may present the selected subset of tiles 1120 for each of the estimated categories 1125 along with confidence scores. For instance, the display 615 may render a GUI with the selected subset of tiles 1120 from the image 1105 along with colors to indicate the associated confidence scores for the estimated categories 1125. The presentation of the information may indicate the tissue type localization as well as region importance scoring.

In this manner, the classification model 635 may be trained to automatically determine categorizations from biomedical images for the samples depicted therein. The learning and training process may be perform from one end (e.g., the tile encoder 650) to the other end (e.g., the tile aggregator 660) of the classification model 635 in one stroke, thereby eliminating any bottlenecks resulting from training individual components of a model separately. Furthermore, the ability to use labels (e.g., the category labels 715) as opposed to annotations of individual pixels in the image to indicate the presence or absence of the condition may alleviate the constraint on training data available for use. Additionally, the feature space defined by a clusterer may be used to deduce parts or latent morphological features embedded in the feature vectors or maps generated by the encoders.

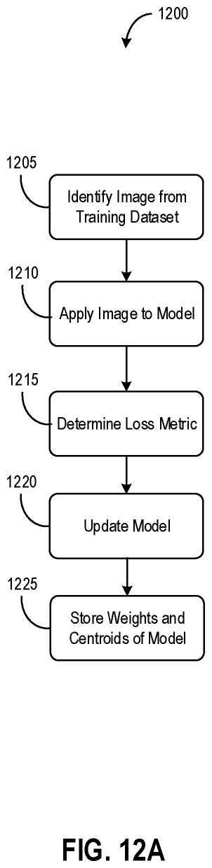

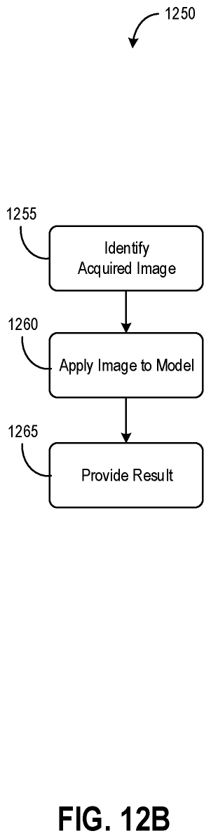

Referring now to FIG. 12A, depicted is a flow diagram depicting a method 1200 of training a model to classify biomedical images using machine learning models. The method 1200 may be implemented using the system 600 described herein in conjunction with FIGS. 6-11 or the system 1300 described herein in conjunction with FIG. 0.13. Under method 1200, a computing system (e.g., the image processing system 605) may identify an image (e.g., the image 705) from a training dataset (e.g., the training dataset 645) (1205). The computing system may apply the image to a model (e.g., the classification model 635) (1210). The computing system may determine a loss metric (e.g., the classification loss metric 1005 and deviation loss metric 1010) (1215). The computing system may update the model (1220). The computing system may store weights and centroids (e.g., the centroids 820) of the model (1225).

Referring now to FIG. 12B, depicted is a flow diagram depicting a method 1250 of classifying biomedical images using machine learning models. The method 1250 may be implemented using the system 600 described herein in conjunction with FIGS. 6-11 or the system 1300 described herein in conjunction with FIG. 0.13. Under method 1250, a computing system (e.g., the image processing system 605) may identify an acquired image (e.g., the image 1105) (1255). The computing system may apply the image to a model (e.g., the classification model 635) (1260). The computing system may provide a result (e.g., the estimated categories 1125) (1265).

### C. Computing and Network Environment

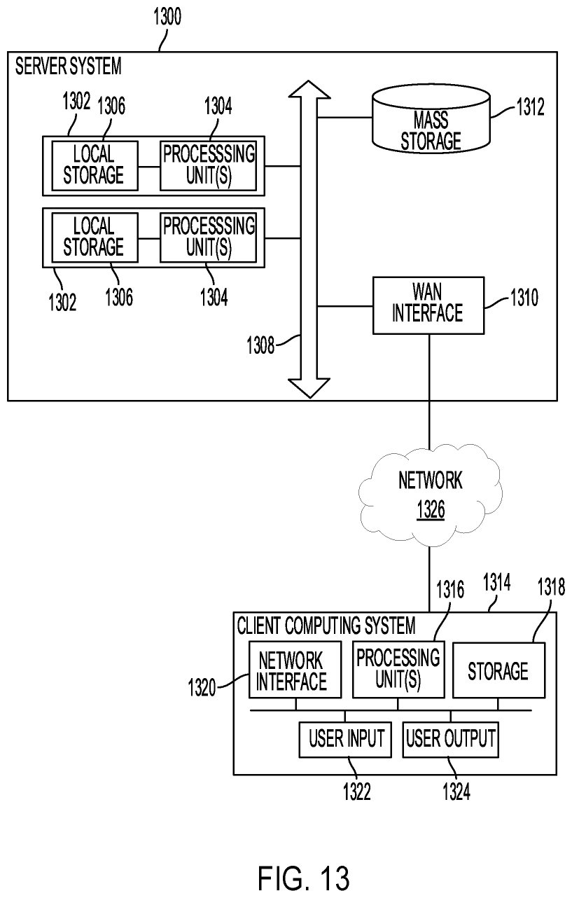

Various operations described herein can be implemented on computer systems. FIG. 13 shows a simplified block diagram of a representative server system 1300, client computer system 1314, and network 1326 usable to implement certain embodiments of the present disclosure. In various embodiments, server system 1300 or similar systems can implement services or servers described herein or portions thereof. Client computer system 1314 or similar systems can implement clients described herein. The system 100 described herein can be similar to the server system 1300. Server system 1300 can have a modular design that incorporates a number of modules 1302 (e.g., blades in a blade server embodiment); while two modules 1302 are shown, any number can be provided. Each module 1302 can include processing unit(s) 1304 and local storage 1306.

Processing unit(s) 1304 can include a single processor, which can have one or more cores, or multiple processors. In some embodiments, processing unit(s) 1304 can include a general-purpose primary processor as well as one or more special-purpose co-processors such as graphics processors, digital signal processors, or the like. In some embodiments, some or all processing units 1304 can be implemented using customized circuits, such as application specific integrated circuits (ASICs) or field programmable gate arrays (FPGAs). In some embodiments, such integrated circuits execute instructions that are stored on the circuit itself. In other embodiments, processing unit(s) 1304 can execute instructions stored in local storage 1306. Any type of processors in any combination can be included in processing unit(s) 1304.

Local storage 1306 can include volatile storage media (e.g., DRAM, SRAM, SDRAM, or the like) and/or non-volatile storage media (e.g., magnetic or optical disk, flash memory, or the like). Storage media incorporated in local storage 1306 can be fixed, removable or upgradeable as desired. Local storage 1306 can be physically or logically divided into various subunits such as a system memory, a read-only memory (ROM), and a permanent storage device. The system memory can be a read-and-write memory device or a volatile read-and-write memory, such as dynamic random-access memory. The system memory can store some or all of the instructions and data that processing unit(s) 1304 need at runtime. The ROM can store static data and instructions that are needed by processing unit(s) 1304. The permanent storage device can be a non-volatile read-and-write memory device that can store instructions and data even when module 1302 is powered down. The term “storage medium” as used herein includes any medium in which data can be stored indefinitely (subject to overwriting, electrical disturbance, power loss, or the like) and does not include carrier waves and transitory electronic signals propagating wirelessly or over wired connections.

In some embodiments, local storage 1306 can store one or more software programs to be executed by processing unit(s) 1304, such as an operating system and/or programs implementing various server functions such as functions of the system 100 of FIG. 1 or any other system described herein, or any other server(s) associated with system 100 or any other system described herein.

“Software” refers generally to sequences of instructions that, when executed by processing unit(s) 1304 cause server system 1300 (or portions thereof) to perform various operations, thus defining one or more specific machine embodiments that execute and perform the operations of the software programs. The instructions can be stored as firmware residing in read-only memory and/or program code stored in non-volatile storage media that can be read into volatile working memory for execution by processing unit(s) 1304. Software can be implemented as a single program or a collection of separate programs or program modules that interact as desired. From local storage 1306 (or non-local storage described below), processing unit(s) 1304 can retrieve program instructions to execute and data to process in order to execute various operations described above.

In some server systems 1300, multiple modules 1302 can be interconnected via a bus or other interconnect 1308, forming a local area network that supports communication between modules 1302 and other components of server system 1300. Interconnect 1308 can be implemented using various technologies including server racks, hubs, routers, etc.

A wide area network (WAN) interface 1310 can provide data communication capability between the local area network (interconnect 1308) and the network 1326, such as the Internet. Technologies can be used, including wired (e.g., Ethernet, IEEE 802.3 standards) and/or wireless technologies (e.g., Wi-Fi, IEEE 802.11 standards).

In some embodiments, local storage 1306 is intended to provide working memory for processing unit(s) 1304, providing fast access to programs and/or data to be processed while reducing traffic on interconnect 1308. Storage for larger quantities of data can be provided on the local area network by one or more mass storage subsystems 1312 that can be connected to interconnect 1308. Mass storage subsystem 1312 can be based on magnetic, optical, semiconductor, or other data storage media. Direct attached storage, storage area networks, network-attached storage, and the like can be used. Any data stores or other collections of data described herein as being produced, consumed, or maintained by a service or server can be stored in mass storage subsystem 1312. In some embodiments, additional data storage resources may be accessible via WAN interface 1310 (potentially with increased latency).

Server system 1300 can operate in response to requests received via WAN interface 1310. For example, one of modules 1302 can implement a supervisory function and assign discrete tasks to other modules 1302 in response to received requests. Work allocation techniques can be used. As requests are processed, results can be returned to the requester via WAN interface 1310. Such operation can generally be automated. Further, in some embodiments, WAN interface 1310 can connect multiple server systems 1300 to each other, providing scalable systems capable of managing high volumes of activity. Other techniques for managing server systems and server farms (collections of server systems that cooperate) can be used, including dynamic resource allocation and reallocation.

Server system 1300 can interact with various user-owned or user-operated devices via a wide-area network such as the Internet. An example of a user-operated device is shown in FIG. 13 as client computing system 1314. Client computing system 1314 can be implemented, for example, as a consumer device such as a smartphone, other mobile phone, tablet computer, wearable computing device (e.g., smart watch, eyeglasses), desktop computer, laptop computer, and so on.

For example, client computing system 1314 can communicate via WAN interface 1310. Client computing system 1314 can include computer components such as processing unit(s) 1316, storage device 1318, network interface 1320, user input device 1322, and user output device 1324. Client computing system 1314 can be a computing device implemented in a variety of form factors, such as a desktop computer, laptop computer, tablet computer, smartphone, other mobile computing device, wearable computing device, or the like.

Processor 1316 and storage device 1318 can be similar to processing unit(s) 1304 and local storage 1306 described above. Suitable devices can be selected based on the demands to be placed on client computing system 1314; for example, client computing system 1314 can be implemented as a “thin” client with limited processing capability or as a high-powered computing device. Client computing system 1314 can be provisioned with program code executable by processing unit(s) 1316 to enable various interactions with server system 1300.

Network interface 1320 can provide a connection to the network 1326, such as a wide area network (e.g., the Internet) to which WAN interface 1310 of server system 1300 is also connected. In various embodiments, network interface 1320 can include a wired interface (e.g., Ethernet) and/or a wireless interface implementing various RF data communication standards such as Wi-Fi, Bluetooth, or cellular data network standards (e.g., 3G, 4G, LTE, etc.).

User input device 1322 can include any device (or devices) via which a user can provide signals to client computing system 1314; client computing system 1314 can interpret the signals as indicative of particular user requests or information. In various embodiments, user input device 1322 can include any or all of a keyboard, touch pad, touch screen, mouse or other pointing device, scroll wheel, click wheel, dial, button, switch, keypad, microphone, and so on.

User output device 1324 can include any device via which client computing system 1314 can provide information to a user. For example, user output device 1324 can include display-to-display images generated by or delivered to client computing system 1314. The display can incorporate various image generation technologies, e.g., a liquid crystal display (LCD), light-emitting diode (LED) including organic light-emitting diodes (OLED), projection system, cathode ray tube (CRT), or the like, together with supporting electronics (e.g., digital-to-analog or analog-to-digital converters, signal processors, or the like). Some embodiments can include a device such as a touchscreen that function as both input and output device. In some embodiments, other user output devices 1324 can be provided in addition to or instead of a display. Examples include indicator lights, speakers, tactile “display” devices, printers, and so on.

Some embodiments include electronic components, such as microprocessors, storage and memory that store computer program instructions in a computer readable storage medium. Many of the features described in this specification can be implemented as processes that are specified as a set of program instructions encoded on a computer readable storage medium. When these program instructions are executed by one or more processing units, they cause the processing unit(s) to perform various operations indicated in the program instructions. Examples of program instructions or computer code include machine code, such as is produced by a compiler, and files including higher-level code that are executed by a computer, an electronic component, or a microprocessor using an interpreter. Through suitable programming, processing unit(s) 1304 and 1316 can provide various functionality for server system 1300 and client computing system 1314, including any of the functionality described herein as being performed by a server or client, or other functionality.

It will be appreciated that server system 1300 and client computing system 1314 are illustrative and that variations and modifications are possible. Computer systems used in connection with embodiments of the present disclosure can have other capabilities not specifically described here. Further, while server system 1300 and client computing system 1314 are described with reference to particular blocks, it is to be understood that these blocks are defined for convenience of description and are not intended to imply a particular physical arrangement of component parts. For instance, different blocks can be but need not be located in the same facility, in the same server rack, or on the same motherboard. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Embodiments of the present disclosure can be realized in a variety of apparatus including electronic devices implemented using any combination of circuitry and software.

While the disclosure has been described with respect to specific embodiments, one skilled in the art will recognize that numerous modifications are possible. Embodiments of the disclosure can be realized using a variety of computer systems and communication technologies including but not limited to specific examples described herein. Embodiments of the present disclosure can be realized using any combination of dedicated components and/or programmable processors and/or other programmable devices. The various processes described herein can be implemented on the same processor or different processors in any combination. Where components are described as being configured to perform certain operations, such configuration can be accomplished; e.g., by designing electronic circuits to perform the operation, by programming programmable electronic circuits (such as microprocessors) to perform the operation, or any combination thereof. Further, while the embodiments described above may make reference to specific hardware and software components, those skilled in the art will appreciate that different combinations of hardware and/or software components may also be used and that particular operations described as being implemented in hardware might also be implemented in software or vice versa.

Computer programs incorporating various features of the present disclosure may be encoded and stored on various computer readable storage media; suitable media include magnetic disk or tape, optical storage media such as compact disk (CD) or digital versatile disk (DVD), flash memory, and other non-transitory media. Computer readable media encoded with the program code may be packaged with a compatible electronic device, or the program code may be provided separately from electronic devices (e.g., via Internet download or as a separately packaged computer-readable storage medium).

Thus, although the disclosure has been described with respect to specific embodiments, it will be appreciated that the disclosure is intended to cover all modifications and equivalents within the scope of the following claims.

What is claimed is:

1. A method of training models to classifying biomedical images, comprising:
identifying, by a computing system, a training dataset comprising (i) a plurality of tiles of a biomedical image of a sample and (ii) a label identifying a first category for the sample;
applying, by the computing system, the plurality of tiles to a classification model, the classification model comprising:
a tile encoder having a first plurality of weights to generate, based on the plurality of tiles, a corresponding plurality of feature vectors defined in a feature space;
a clusterer to select a subset of feature vectors from the plurality of feature vectors based on a plurality of centroids defined in the feature space; and
an aggregator having a second plurality of weights to determine, based on the subset of feature vectors, a second category for the sample;
determining, by the computing system, a loss metric based on a comparison between the second category determined by the classification model with the first category of the label of the training dataset;
updating, by the computing system using the loss metric, at least one of the first plurality of weights in the tile encoder, the plurality of centroids of the clusterer, or the second plurality of weights in the aggregator based on the comparison; and
storing, by the computing system, in one or more data structures, the first plurality of weights in the tile encoder, the plurality of centroids defined by the clusterer, and the second plurality of weights of the aggregator.

2. The method of claim 1, wherein applying further comprises applying the plurality of tiles from the biomedical image of a plurality of biomedical images to the classification model, the clusterer of the classification model to identify the plurality of feature vectors from which to select the subset of feature vectors based on the biomedical image.

3. The method of claim 1, wherein applying further comprises applying the plurality of tiles to the classification model, the clusterer of the classification model to select a subset of tiles in the biomedical image based on the subset of feature vectors.

4. The method of claim 1, wherein applying further comprises applying the plurality of tiles to the classification model, the aggregator of the classification model to determine a plurality of confidence scores for a subset of tiles corresponding to the subset of feature vectors.

5. The method of claim 1, wherein identifying further comprises identifying the training dataset comprising a plurality of labels for a corresponding first plurality of categories for the sample, and
wherein applying further comprises applying the plurality of tiles to the classification model, the aggregator of the classification model to determine a second plurality of categories for the sample.

6. The method of claim 1, further comprising determining, by the computing system, a second loss metric based on comparison among the plurality of feature vectors and the plurality of centroids in the feature space, and
wherein updating further comprises updating, using the second loss metric, at least one of the first plurality of weights in the tile encoder or the plurality of centroids of the clusterer within the feature space.

7. The method of claim 1, wherein updating further comprises determining the plurality of centroids based on a second plurality of feature vectors generated by the tile encoder, subsequent to updating of the first plurality of weights.

8. A method of classifying biomedical images, comprising:
identifying, by a computing system, a first plurality of tiles from a first biomedical image of a first sample;
determining, by the computing system, a first category for the first sample by applying the plurality of tiles to a classification model, the classification model trained using a training dataset having a plurality of examples each including (i) a second plurality of tiles of a second biomedical image of a second sample and (ii) a label identifying a second category for the sample, the classification model comprising:
a tile encoder having a first plurality of weights to determine, based on the first plurality of tiles, a corresponding plurality of feature vectors in a feature space;
a clusterer to select a subset of feature vectors from the plurality of feature vectors based on a plurality of centroids defined in the feature space; and
an aggregator having a second plurality of weights to generate, based on the subset of feature vectors, the first category for the sample,
storing, by the computing system, an association between the first category and the first biomedical image.

9. The method of claim 8, further comprising providing, by the computing system, the association between the first category and the first biomedical image.

10. The method of claim 8, wherein determining further comprises determining a plurality of confidence scores for a subset of tiles corresponding to the subset of feature vectors.

11. The method of claim 8, wherein determining further comprises determining a second plurality of categories for the sample.

12. The method of claim 8, wherein determining further comprises selecting a subset of tiles in the biomedical image based on the subset of feature vectors.

13. The method of claim 8, wherein identifying further comprises selecting the first plurality of tiles from a second plurality of tiles of the first biomedical image.

14. The method of claim 8, further comprising obtaining, by the computing system, the first biomedical image of the first sample via a histological image preparer.

15. A system for classifying biomedical images, comprising:
a computing system having one or more processors coupled with memory, configured to:
identify a first plurality of tiles from a first biomedical image of a first sample;
determine a first category for the sample by applying the plurality of tiles to a classification model, the classification model trained using a training dataset having a plurality of examples each including (i) a second plurality of tiles of a second biomedical image of a second sample and (ii) a label identifying a second category for the sample, the classification model comprising:
a tile encoder having a first plurality of weights to determine, based on the first plurality of tiles, a corresponding plurality of feature vectors in a feature space;
a clusterer to select a subset of feature vectors from the plurality of feature vectors based on a plurality of centroids defined in the feature space; and
an aggregator having a second plurality of weights to generate, based on the subset of feature vectors, the first category for the sample,
store an association between the first category and the first biomedical image.

16. The system of claim 15, wherein the computing system is further configured to provide the association between the first category and the first biomedical image.

17. The system of claim 15, wherein the computing system is further configured to determine a plurality of confidence scores for a subset of tiles corresponding to the subset of feature vectors.

18. The system of claim 15, wherein the computing system is further configured to a second plurality of categories for the sample.

19. The system of claim 15, wherein the computing system is further configured to select a subset of tiles in the biomedical image based on the subset of feature vectors.

20. The system of claim 15, wherein the computing system is further configured to select the first plurality of tiles from a second plurality of tiles of the first biomedical image.
